SEQUENCE LISTING (2 sequences)

SEQ ID NO: 1 - DNA, 12 bp, Artificial Sequence
  synthetic
agannnnnnctg

SEQ ID NO: 2 - DNA, 39 bp, Artificial Sequence
  primer
atagctagaatccgattactaagatcgaagatcgactcg

(19) United States
(12) Patent Application Publication
(10) Pub. No.: US 2003/0104428 A1
(43) Pub. Date: Jun. 5, 2003

(54) Method for characterization of nucleic acid molecules

(75) Inventors: Daniel Branton, Lexington, MA (US); Stephen S. Laderman, Menlo Park, CA (US); Zohar Yakhini, Zikhron Ya'acov (IL); Jeffrey R. Sampson, San Francisco, CA (US); Hui Wang, Cambridge, MA (US)

Correspondence Address: HALE AND DORR, LLP, 60 STATE STREET, BOSTON, MA, 02109

(73) Assignee: President and Fellows of Harvard College, Cambridge, MA (US)

(21) Appl. No.: 10/177,062

(22) Filed: Jun. 21, 2002

Related U.S. Application Data
(60) Provisional application No. 60/299,878, filed on Jun. 21, 2001.

(51) Int. Cl. C12Q 1/68; C07H 21/04; C12P 19/34

(52) U.S. Cl. 435/6; 435/91.2; 536/23.1

(57) ABSTRACT

To probe the characteristics of a target nucleic acid molecule or a population of target nucleic acid molecules, a local cross-sectional area, local charge, or local chemistry of a molecule is modified. The modified nucleic acid is contacted with a substrate that includes a detector that is responsive to the modification in the local cross-sectional area, local charge, or local chemistry of the nucleic acid molecule. The modified nucleic acid molecule traverses a defined and preferably molecular dimensioned volume on the substrate so that nucleotides of the modified nucleic acid molecule interact with the detector in sequential order, whereby data correlating with the cross-sectional area, local charge, or local chemistry of the nucleic acid molecule are obtained. The nucleic acid molecule may be modified in a number of locations along the molecule's length. Because the individual nucleotides of the nucleic acid molecule interact with the detector in sequential order, information regarding the location and composition of a plurality of modified sites along a single molecule can be obtained.

### RELATED APPLICATION

This application claims the benefit of U.S. Provisional Application Serial No. 60/299,878, filed Jun. 21, 2001, the entire contents of which are incorporated herein.

### STATEMENT AS TO FEDERALLY SPONSORED RESEARCH

This invention was made, in part, with United States Government support under DARPA grant number N65236-98-1-5407. The Government has certain rights in this invention.

### BACKGROUND OF THE INVENTION

The present invention relates to the field of detecting and identifying genetic materials, such as nucleic acid molecules of interest. In particular, the invention relates to methods for detection of alleles and haplotypes, and to methods for detecting messenger RNAs, including alternate splice forms.

DNA sequence information has revolutionized the pharmaceutical and medical industries. In particular, the measurement of DNA sequence variations, termed genotyping, is becoming a well-established method for locating disease-causing or disease-associated genes from which new potential drug targets can be identified (Editorial, (1996) Nat Biotechnol 14, 1516-1518; Persidis, A. (1998) Nat Biotechnol 16, 791-2; and Ball, S. and Borman, N. (1997) Nat Biotechnol 15, 925-6). Although this process, termed pharmacogenetics, has been used to identify less than 10% of the current drug targets in the pharmaceutical pipeline, it promises to have a great impact on the future of the drug discovery process.

Genotyping using single nucleotide polymorphism (SNP) markers is becoming the method of choice for performing disease association studies (Wang, D. G., et al. (1998) Science 280, 1077-82; and Schafer, A. J. and Hawkins, J. R. (1998) Nat Biotechnol 16, 33-9). It is estimated that there exist between one and three million SNPs in the human genome. Disease association studies require the analysis of large numbers of SNPs (1,000s) on a relatively large number of individuals (1,000s). It is likely that finer SNP mapping will require studies utilizing greater than 10,000 SNPs. This translates into millions of SNP analyses per study.

SNP genotyping can be also used for patient stratification during clinical trials to better assess drug efficacy, toxicity and dosing. Although patient stratification will most likely require the analysis of a fewer number of SNPs (low 1,000s), a larger number of individuals (10,000s) is required, which also translates into millions of analyses per study. Fewer SNPs need be analyzed in this case because the diagnostic SNPs will have been identified during the previous drug discovery study. A large number of individuals are needed to stratify patients from a very broad population range in order to understand the full range of responses in a diverse population.

As the pharmacogenomic approach to medicine materializes, it will become common practice to genotype individuals for those SNPs which are diagnostic for genetic disease, disease predisposition, drug efficacy, and drug toxicity. This is likely to require the analysis of a few 1,000 SNPs in 10s of thousands of individuals. This translates into millions of analyses. Most importantly, these types of applications will require methods that are extremely accurate and robust and fully integrated into easy-to-use measurement systems.

A number of techniques have been employed to detect allelic variants of genetic loci including analysis of restriction fragment length polymorphic (RFLP) patterns, use of oligonucleotide probes, and DNA amplification methods. Most of the current methods have throughputs between 10,000 and 100,000 analyses per 20-hour day per system. For coarse mapping studies, which require between 100,000 and 1 million analyses per study, the current systems are adequate since they could complete an analysis in one week or less. However, as the pharamcogenomic approach to drug development materializes, and both the size (total number of analyses) and number of association studies increase, the resulting 10s of millions of allele-calls needing to be determined will require much higher throughput systems in order to complete the studies in a reasonable length of time.

A disadvantage of many current methods is that they require the sequence complexity of the target sample to be reduced and the copy number of the target sequence to be amplified. This can be accomplished using either the polymerase chain reaction (PCR), ligase chain reaction (LCR) or rolling circle replication methods (U.S. Pat. No. 5,854,033). Moreover, many of the current genotyping methods are best performed using single stranded nucleic acid targets, which then require an additional post-amplification step. These two limitations not only reduce the overall sample throughput through increased sample handling steps, but also increase the analyses cost through increased reagent and disposable plastic use and requirement of additional sample handling instrumentation.

Finally, none of the current methods are capable of directly determining the genetic haplotype of a sample. It is becoming clear from genetic studies that defined combinations of SNPs are responsible for, or are closely linked with, the disease-causing loci or genes. Thus, knowing which specific genetic alleles reside together on a single distinct chromosome is becoming increasingly important. To date, the only way to accomplish this task is to physically separate the two chromosomes within the genomic sample using some type of artificial cloning scheme prior to the allele analysis. This step is both time consuming and requires additional reagents which reduces the sample throughput and increases the overall analysis cost.

Other applications make it desirable to quantify the absolute or relative number of a particular polymer type in a mixture containing other nucleic acid polymers. For example, it may be important to detect a particular DNA type found in a pathogenic bacteria in an environmental sample that contains many other DNA or RNA polymers. Or it may be desirable to detect and quantify certain mRNAs in a cell sample since the levels of different mRNAs in the cell, at any given time, provide valuable information about the ongoing cellular metabolism. For example, detection of specific mRNAs has been pivotal in the studies of gene activation and identification of pathologies and latent infections.

Nanopore technology can be used to detect and count single nucleic acid molecules at a very high rate. It can also detect differences in polymer length, composition and structure. Church et al. in U.S. Pat. No. 5,795,782 report that a voltage bias can drive single-stranded charged polynucleotides through a 1-2 nanometer transmembrane channel in a lipid bilayer. Data in the form of variations in channel ionic current provide insight into the characterization and structure of biopolymers at the molecular and atomic levels. The passage of an individual strand through the channel is observed as a transient decrease in ionic current. It also has been observed that the current blockage caused by polymer translocation is sensitive to local cross-sectional volume occupied by the polymer. The above demonstrations were performed using the natural α-hemolysine channel protein from Staphylococcus aureus embedded in a synthetic lipid bilayer membrane. This pore has a limiting aperture which accommodates single stranded DNA but excludes double stranded DNA. See, U.S. Pat. No. 5,795,782 and Kasianowicz et al. (“Characterization of individual polynucleotide molecules using a membrane channel”, Proc. Natl. Acad. Sci. 93:13770 (November 1996)). Thus, application of technology is limited.

Although it is clear that the analysis of genetic material can be used to identify an organism or infectious agent, the challenge is to develop methods with the necessary speed, robustness, sensitivity and universality to perform the analysis outside of the research laboratory setting. A high-throughput device that can probe and directly read, at the single-molecule level, hybridization state, base stacking, and sequence of a cell's key biopolymers such as DNA, RNA and even proteins, will dramatically alter the pace of biological development.

### SUMMARY OF THE INVENTION

In one aspect of the invention, a method is provided for characterizing a nucleic acid molecule. In this aspect of the invention, a property of at least one defined local area of the nucleic acid molecule is modified. By “defined local area,” is meant herein a nucleic acid sequence comprising fifty or fewer consecutive nucleotides. In some embodiments of the invention, the defined local area comprises thirty or fewer consecutive nucleotides. In some other embodiments of the invention, the defined local area comprises twenty or fewer consecutive nucleotides. In some other embodiments of the invention, the defined local area comprises ten or fewer consecutive nucleotides. In at least some embodiments of the invention, the defined local area is modified by altering the local cross-sectional area of at least one nucleotide. In one or more embodiments, the local cross-sectional area includes characteristics such as local bulk, charge, and/or charge density. The modified nucleic acid is contacted with a substrate that includes a detector that is responsive to the modification in the local cross-sectional area, local charge, or local chemistry of the nucleic acid molecule. The modified nucleic acid molecule traverses a defined and preferably molecular dimensioned (e.g. very small) volume on the substrate so that nucleotides of the modified nucleic acid molecule interact with the detector in sequential order, whereby data correlating with the cross-sectional area, local charge, or local chemistry of the nucleic acid molecule are obtained. The nucleic acid molecule may be modified in a number of locations along the molecule's length. Because the individual nucleotides of the nucleic acid molecule interact with the detector in sequential order, information regarding the location and composition of a plurality of modified sites along a single molecule can be obtained.

In one or more embodiments of the invention, two or more defined local areas are modified. Modification of the defined local area is used herein to refer a detectable change in the molecule profile in a defined region or volume of the molecule. The modification can alter the local bulk, charge, charge density, or chemistry of the molecule, or it can alter an electronic property of the molecule. The modification can be accomplished using a variety of methods, including the introduction of an identifier at the local site. Exemplary identifiers include chemical reagents that react at the local site, enzymes that introduce modifications at the local site, binding agents, and probes such as oligonucleotides and sequence-specific binding proteins. Modification of the nucleic acid also includes the introduction of a modified nucleotide monomer in the nucleic acid polymer to be characterized. The modifying step can be accomplished by chemically modifying a nucleotide of the nucleic acid molecule, or altering its local charge. The chemical modifier may include a covalently linked moiety capable of generating an identifiable signal upon interaction with the detector. The modifying step can be accomplished by non-covalently binding a probe to the nucleic acid molecule. In one or more embodiments, the probe is an oligonucleotide, or a sequence-specific protein. In some embodiments, the sequence-specific protein is a Zn²⁺ finger protein or other DNA-binding motif, as are commonly found, for example, in transcription factors.

In some embodiments, the nucleic acid molecule is single stranded, or it is double stranded. In some other embodiments, the nucleic acid molecule is in a sample of genomic DNA. In some embodiments, the probe binds a specific nucleic acid sequence. In one or more embodiments, the sequence contains a single nucleotide polymorphism (SNP) locus or many single nucleotide polymorphism loci.

In another aspect of the invention, a method for characterizing a nucleic acid molecule includes providing (i) a sample comprising at least one nucleic acid molecule; and (ii) a probe capable of binding to a specific nucleic acid sequence; and combining the sample and the probe under conditions such that the probe binds to or modifies the nucleic acid molecule to form a nucleic acid:probe complex. The presence and location of binding in the nucleic acid:probe complex is detected by contacting the nucleic acid:probe complex with a substrate, the substrate including a detector capable of identifying a characteristic of a nucleic acid molecule; and causing the nucleic acid:probe complex to traverse a defined volume of the substrate, preferably molecular dimensioned (e.g. very small) volume, so that nucleotides of the nucleic acid interact with the detector in sequential order, whereby data correlating with the presence and/or location of binding or modification are obtained.

In some embodiments, the probe comprises a sequence-specific binding protein, such as a Zn²⁺ finger protein or other DNA binding motif. In other embodiments, the probe may be an oligonucleotide, for example, a genome-specific oligonucleotide, an allele-specific oligonucleotide, or a set of oligonucleotides having universal properties.

In another aspect of the invention, a method for characterizing a nucleic acid molecule includes providing (i) a sample comprising at least one nucleic acid molecule, and (ii) a plurality of identifiers, each identifier capable of binding to or modifying a specific nucleic acid sequence, and combining the sample and the identifiers under conditions where the connectivity of the nucleic acid molecule is maintained so that long pieces of nucleic acid molecules, greater than 1000 bp, and preferably greater than 20,000 bp long are maintained.

In one or more embodiments, the identifier is a hybridizable probe. The probe binds to or modifies the nucleic acid molecule to form a nucleic acid:probe complex at locations where the specific nucleic acid sequence is present. The presence and location of binding in the nucleic acid:probe complex is detected by contacting the nucleic acid:probe complex with a substrate, the substrate including a detector capable of identifying a characteristic of a nucleic acid molecule, or a characteristic of the probe, or a characteristic of the nucleic acid: probe complex, and causing the nucleic acid:probe complex to traverse a defined volume of the substrate, preferably molecular dimensioned (e.g. very small) volume, so that nucleotides of the nucleic acid interact with the detector in sequential order, whereby data correlating with the presence and/or location of binding or modification are obtained. The relationship between multiple loci on one nucleic acid molecule, or among different nucleic acid molecules in a mixture is established by identifying and distinguishing among the different nucleic acid:probe complexes in the mixture. Information that can be obtained from a nucleic acid molecule or population of nucleic acid molecules population of nucleic acid molecules includes the number of a selected nucleic acid:probe or identifier complexes, and the relative location or locations of probe or identifier binding on a nucleic acid molecule.

In another aspect of the invention, a method is provided for detection of at least one allele of a genetic locus. The modified local defined area of the nucleic acid molecule may correspond to a specific nucleotide sequence of the nucleic acid molecule. Thus, observation of a region of modified local defined area is directly correlated to the presence of a specific nucleotide sequence in the sample. Because multiple allele sites may be detected on a single nucleic acid molecule, the method is ideally suited for the direct determination of the genetic haplotype of the sample.

In another embodiment of the invention, an assay is provided for SNP genotyping analyses using DNA, e.g., native double-stranded genomic DNA or double-stranded DNA fragments obtained by conventional amplification methods. The assay selects one or more zinc finger protein(s) (ZFP) to bind to a defined region or regions of a double-strand DNA containing a sequence of interest. This method enables the direct detection of sequence variations in double stranded DNA molecules, which enables complex genetic haplotypes of a genomic sample to be easily determined. In another embodiment of the invention, a method is provided for detecting messenger RNAs, including alternate splice forms. The method may also be used to determine expression levels of mRNA or to identify regions of genetic material associated with specific cellular functions.

In another aspect of the invention, a method for characterizing a nucleic acid molecule includes generating a population of double stranded nucleic acids fragments of differing lengths from a target double stranded nucleic acid, and characterizing the nucleic acid fragments by contacting the nucleic acid fragment population with a surface, the surface including a detector capable of detecting the presence of a nucleic acids and causing the nucleic acid fragments to traverse a defined volume on the solid state substrate so that the nucleotides of a nucleic acid fragment interact with the detector in sequential order, whereby data correlated with a characteristic of the nucleic acid fragment are obtained. The method may be used to determine the relative amount and/or length of the fragments. The method may also be used to determine a size distribution of nucleic acid fragments.

In another aspect, the invention provides a method for characterizing a nucleic acid molecule by modifying at least electronic property of the nucleic acid molecule by modifying at least one nucleotide of the molecule. The modified nucleic acid molecule is contacted with a substrate that includes a detector. The detector is capable of identifying the modification of the electronic property of the nucleic acid molecule when the molecule traverses a defined volume on the substrate, so that individual nucleotides of the nucleic acid molecules interact with the detector in sequential order. Data correlating with the modification of the electronic property of the nucleic acid molecule are obtained. In at least some embodiments, the detector identifies a current tunneling characteristic of the nucleic acid. Modifications that alter the electronic property of the nucleic acid molecule include, but are not limited to, introduction of charged atoms or molecules into the nucleic acid molecule, for example, bromine and other halogens, addition of bulky chemical groups, including, but not limited to alkyl groups, binding of oligonucleotide probes, binding of sequence-specific DNA or RNA binding proteins, addition of bulky tags such as biotin, streptavidin, and other modifications of nucleotides and nucleic acids known in the art.

The nanoscale devices and methods of their use in the present invention possess particular demonstrated capabilities that are well-suited for the methods described above. First, characteristic features of the translocating polymer are directly converted into an electrical signal. Transduction and recognition occur in real time, on a molecule-by-molecule basis. Second, a nanopore is a single molecule detector, but it functions as a high throughput device. Thousands of different molecules or thousands of identical molecules can be probed in a few minutes. Third, channel blockage is sensitive to the local cross-sectional area of the molecule. When polymers whose cross-sectional area is increased by secondary structure translocate through the pore, more of the current is blocked (less current flows) than when a strand lacking such secondary structure translocates through the pore. Fourth, long, continuous segments of DNA can be probed. Although practical considerations may limit the length of DNA that is detected as it translocates through a nanopore, we are not aware of any theoretical limits.

The term “binding” is used broadly to refer to any mode of affinity or adherence a molecule or probe may have for a substrate, such as a target nucleic acid. Binding of nucleic acids typically occurs at a location where the shape and chemical natures of the respective molecule surfaces are complementary. For example, proteins, such as ZFPs, will preferentially bind to sequence-specific regions of a nucleic acid, where the shape and chemical nature of the respective molecule surfaces favor binding.

The term “probe” as used herein refers to any molecule or plurality of molecules each having a binding affinity for at least one target nucleic acid sequence or target nucleic acid structure when the binding site is present in the nucleic acid molecule.

A nucleic acid is a linear polymer, although it may have more complex secondary or tertiary structures. The term “sequential order” is used to indicate that the nucleic acid is probed in linear, or extended, form so that each individual nucleotide interacts with the detector in order of its appearance along the length of the nucleic acid. As used herein a “nucleic acid” includes any linear sequence of nucleotides, such as DNA, e.g., single and double stranded DNA, genomic DNA, or cDNA and RNA, e.g., genomic RNA, mRNA, fragments thereof, and DNA-RNA hybrid molecules thereof. Although the nucleic acid molecules may in vivo be associated with other complexing molecules, e.g., proteins, such complexing molecules typically are removed prior to characterization. Thus, reference in the description to “DNA” is not intended to be limiting, and it is understood that the above and other art recognized nucleic acid moieties may be characterized using the method of the invention.

The term “contacting a nucleic acid with a substrate,” encompasses causing the nucleic acid and the substrate to be brought into direct physical contact or into close proximity, particularly nanoscale or subnanoscale proximity. For example, the nucleic acid and the substrate are in contact when the nucleic acid is traversing a nanopore or other channel within the substrate, or when the nucleic acid is traversing a groove in the surface of the substrate.

The term “defined volume of a substrate” refers to a region, preferably a molecularly sized volume of space, in or on the substrate to which the target nucleic acid molecules are confined during characterization according to the method of the invention. A nanopore represents an example of a molecularly dimensioned pore or channel that provides a defined volume. For simplification, the term “nanopore” is most widely used throughout the specification when referring to detection and characterization of nucleic acid molecules, however, it is understood that a nanopore is merely an example of a defined volume of the invention. The defined volume includes other physical barriers, such as a channel or groove in a substrate, or it may arise using other means, such as an electric field, or concentration gradient.

The term “allele,” as used herein, means a genetic variation of a nucleic acid sequence. The variation may be associated with a coding region; that is, an alternative form of the gene. Alternatively, the variation may occur in regions of DNA that are not coding. The use of the term allele should be interpreted broadly to include both coding and non-coding regions of the DNA sequence. An “allele” may be viewed as a subset of sequence variations including, but not limited to, “single nucleotide polymorphisms” or SNPs, deletions, insertions, and variations in length and number of repeated sequences.

As used herein, “haplotype” is set of alleles on one chromosome or a part of a chromosome that are usually inherited as a unit, i.e. the genes are linked.

The term “linkage,” as used herein, refers to the degree to which regions of genomic DNA are inherited together. Regions on different chromosomes do not exhibit linkage and are inherited together 50% of the time. Adjacent genes that are always inherited together would be said to exhibit 100% linkage. Other degrees of linkage are possible when genes are located on the same chromosome but spaced some distance apart. Such genes exhibit linkage between 50% and 100%.

As used herein, the terms “endonuclease” and “restriction endonuclease” refer to an enzyme that cuts double-stranded DNA having a particular nucleotide sequence. The specificities of numerous endonucleases are well known and can be found in a variety of publications, e.g. Molecular Cloning: A Laboratory Manual by Maniatis et al, Cold Spring Harbor Laboratory 1982. That manual is incorporated herein by reference in its entirety.

The term “restriction fragment length polymorphism” (or RFLP), as used herein, refers to differences in DNA nucleotide sequences that produce fragments of different lengths when cleaved by a restriction endonuclease.

The term “primer-defined length polymorphisms” (or PDLP), as used herein, refers to differences in the lengths of amplified DNA sequences due to insertions or deletions in the region of the locus included in the amplified DNA sequence.

The term “Zn²⁺ finger protein” (or ZFP), as used herein, refers to any peptide, polypeptide, or protein comprising an amino acid sequence that comprises a minimal zinc finger motif. As used herein, Zn²⁺ finger proteins encompass naturally occurring Zn²⁺ finger proteins as well as genetically engineered Zn²⁺ finger proteins.

### BRIEF DESCRIPTION OF THE DRAWINGS

The invention is described with reference to the figures, which are presented for the purpose of illustration only and are not limiting of the invention.

FIG. 1 shows translocation current signatures of dA100 at 22° C. This figure shows two translocation events, in which each drop in current corresponds to a single dA100 molecule.

FIG. 2 illustrates the translocation of a nucleic acid molecule for which regions are hybridized with an oligonucleotide.

FIG. 3A is an illustration of encoding and translocation of genetic materials through a nanopore detector according to the invention; FIG. 3B shows a current trace of the genetic material as it traverses the nanopore. The initial current drop is an indication that the molecule has entered the pore, while subsequent larger current drops reflect the passage of the oligonucleotide-hybridized regions; FIG. 3C is another illustration of encoding and translocation of genetic materials through a nanopore detector according to the invention.

FIG. 4 is an illustration of SNP identification using zinc finger proteins (ZFPs) as the marker in the present invention. In FIG. 4A, A ZFP:DNA complex is formed for that allele containing the appropriate sequence variant, while in FIG. 4C, the DNA does not form ad ZFP:DNA complex. FIGS. 4B and 4D show the respective current traces of the complexed and uncomplexed DNA molecules in the sample.

FIG. 5 illustrates the determination of multiple DNA samples using the method of the invention. A particular DNA is identified by the distance (time) of the current drop from the time the DNA enters the nanopore.

FIG. 6 illustrates yet another embodiment of the invention in which additional ZFPs are selected to specifically bind the fragments at defined locations, which will result in an identifiable “coded” pattern for each fragment within the sample mixture.

FIG. 7 illustrates the use of oligonucleotide ligation in the characterization of nucleic acid molecules according to the invention.

FIG. 8 demonstrates the destabilizing effect of an unstructured nucleic acid (UNA) nucleotide analogue base-pair.

FIG. 9 illustrates the complexing of oligonucleotide probes to RNA molecules of interest and their identification including the possibility of identifying their specific splice form based upon the resulting unique current signal of each pattern of complexation

FIG. 10 is an illustration of restriction fragment length polymorphism (RFLP) analysis according to the method of the present invention.

FIG. 11 is an illustration of a sequence fragment (solid horizontal line) containing 2 SNPs (two vertical lines), separated by distance Δ_{I}. Specific ZFPs are represented as dotted lines. The physical distance between Φ₁ and Φ₂ in base pairs is designated as Δ_{I}. Brackets on the sequence fragment represent degree of error in measuring the distance between Φ₁ and Φ₂.

FIG. 12 is an illustration of SNP labeling and assay of four possible haplotypes.

FIG. 13 is an illustration of the total number of SNP loci that can be probed in one assay with a set of ZFPs.

### DETAILED DESCRIPTION OF THE DISCLOSURE

In one or more embodiments, the present invention discloses the use of nanopores for the detection, identification and quantification of one or many different DNA or RNA molecules in a mixture. The mixture may be highly complex and may contain two or more different types of DNA or RNA molecules. The nanopore detection scheme of the invention permits identification and quantification of specific types of single DNA and RNA molecules as they translocate through a defined, preferably molecularly-dimensioned volume of space and interact at the detector in a linear, single-molecule manner. Detection and quantification can be obtained with high precision from extremely small samples and/or relatively dilute or low-abundance polynucleotide samples. The invention also provides for the detection of at least one allele of a genetic locus, and also provides direct determination of the genetic haplotype.

In one or more embodiments, the method of the present invention is carried out using an apparatus that includes a surface having a defined volume located therein, such as groove or aperture defining a channel, passageway or other opening. Either a proteinaceous or a solid-state nanopore can be used to establish a defined volume. A detector is used to identify time-dependent current variations, and therefore nucleotide-dependent, interactions of the molecule with the aperture. Additionally, an amplifier or recording mechanism may be used to detect changes in the ionic or electronic conductances across the aperture as the polymer traverses the opening. The detection method is sensitive enough to discriminate, as needed, between different types of molecules, preferably on a single-molecule level, and/or between regions of varying molecular size or bulk or other features such as charge density. In addition, the method effectively concentrates the target molecule at the detector.

At least two modes of detection are useful in characterizing nucleic acid molecules according to the invention. The first type measures the ion flow through the channel. For this type of detection, a constraining or limiting diameter of the channel is the detector. The constraining diameter can be a feature of the aperture, or it can arise from a molecule of biological origin positioned at, adjacent to, bordering, or within the aperture (that has been suitably linked to the aperture). In one or more embodiments, the channel itself may include a constraining diameter that occupies a length of the channel that is commensurate with the distance between monomers, e.g. nucleic acids, and which is of a dimension on the order of the monomer size, so that conductivity is modulated by the molecular interactions of each successive monomer.

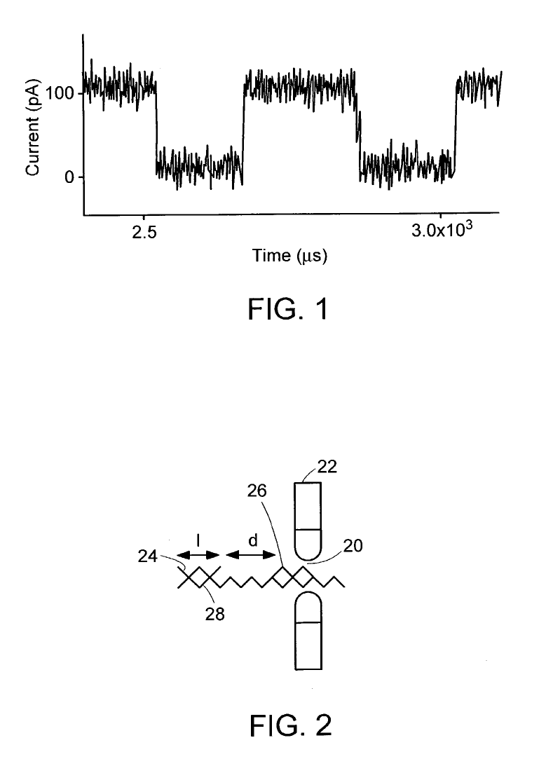

When an appropriate voltage bias is applied so as to create the appropriate driving force, for example, across a membrane containing a nanometer-sized aperture, nucleic acids will traverse the aperture in sequential, monomer order. In this example using the first mode of detection cited above, the nanometer-sized aperture, or nanopore constitutes a defined small volume of space through which the polymer translocates. As the nucleic acid traverses the nanopore, the nucleic acid polymer, and more particularly any attached local labels or identifiers, partially or totally block the current flow through the defined volume of space. Thus, in this first mode of detection, generally, each translocation event, and more particularly any attached local labels, is distinctly observed as a drop in current to a constant fraction of the open pore current, as is illustrated in FIG. 1.

A second mode of detection, according to one or more embodiments of the invention, measures electron flow across the aperture diameter or across its length using nanofabricated electrodes suitably placed at the aperture entrance and/or exit. In this embodiment, first and second electrodes adjacent to or bordering the aperture serve as detectors. The electrodes are positioned so as to monitor the candidate polymer molecules that translocate the aperture. Asperities or constraining dimensions defined by the electrode edge or tip provide suitably dimensioned detectors, as they do in scanning tunneling microscopy. The interested reader is directed to co-pending application U.S. Ser. No. 09/602,650, filed Jun. 22, 2000, the contents of which are incorporated in its entirety by reference, for further details of the apparatus and methodology.

In the second detection mode cited above, as the nucleic acid traverses the nanopore or small volume of space between suitably placed electrodes, the nucleic acid polymer, and more particularly any attached local labels, modify the current, or voltage, or capacitance between the electrodes. In the second mode of detection, each translocation event will be seen as a change in the current or the voltage or the capacitance between the two electrodes. The duration of the current drop or electronic property change of the small volume of space is proportional to polymer length and the degree of current drop or electronic property change can, in part, depend upon the polymer composition (NA sequence composition). See, U.S. Pat. No. 5,795,782 and U.S. Pat. No. 6,015,714.

For both modes of detection, translocation typically occurs within micro to millisecond time scales. For example, in the α-hemolysin channel, the most probable translocation time at 20° C. is 330 μsec for a 100-mer of polydeoxyadenylic acid (dA₁₀₀) and 120 usec for a 100-mer of polydeoxycytidylic acid (dC₁₀₀). DNA of mixed sequences has translocation durations that fall between poly-dA and poly-dC. See, FIG. 1.

Since the blockage or electronic properties of the defined small volume of space between electrodes that is caused by the nucleic acid is sensitive to the local cross-sectional area and or electrical properties occupied by the polymer, covalently or noncovalently attached labels that add to the local cross-sectional area or change the local electrical properties of the polymer can cause an additional blockage or modification of local current signal of the nanopore. The changes in the translocation current signature correspond to the locations of the modifications along the DNA strand. Thus, even without achieving single-base resolution, polymers that have been modified can be distinguished from unlabelled molecules which do not show modified current signatures. This identification is possible because the nanopore is capable of providing information about local nucleotide modifications, i.e., defined local area, together with information about polymer length (including length between local base modifications) and the relative number of such molecules in the mixture on a molecule-by-molecule basis.

Modification of the local cross-sectional area of a nucleic acid molecule may be accomplished in many ways. For example, bulk may be added to the molecule by non-covalent binding of oligonucleotides or sequence-specific binding proteins to discrete regions of the target nucleic acid molecule. Changes in bulkiness of the nucleic acid can also include segements of abasic regions that reduce the local cross-sectional area of the translocating polymer. Alternatively, unique identifiers may be covalently attached to the nucleic acid. The identifier may be a chemical moiety that generates a unique and identifiable ion current signature in the nanopore.

The present invention is described in detail with reference to haplotyping and ionic flow measurements as an example of the overall approach and informatic considerations that must be taken into account in identifying and characterizing nucleic acids. It is recognized, however, that the method of the invention can be used to obtain other information about nucleic acid molecule. Furthermore, modifications in the detection method are contemplated as part of the present invention.

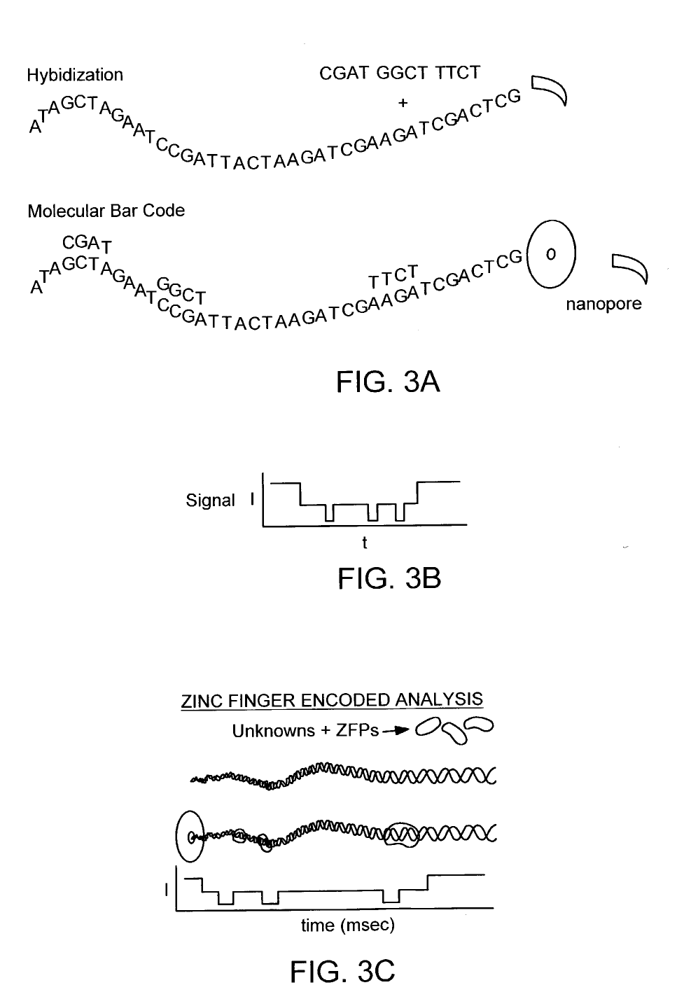

Use of the present invention to haplotype DNA is illustrated in FIG. 3. FIG. 3A illustrates haplotyping using an oligonucleotide probe, and FIG. 3C illustrates haplotyping using a Zn²⁺ finger protein as a probe. The Figure illustrates that the pore diameter is large enough to admit the DNA and its bound label, yet small enough to force the bases of the polynucleotide to traverse in single-file order. In the case of haplotyping using double stranded DNA with bound ZFPs, the pore should have a diameter of about 3-4 nm, which is larger than the aperture provided by channel proteins. Smaller pores (1.5-2.5 nm) will be required for work with double stranded nucleic acids and larger pores (4-5 nm) may be required when working with double stranded DNA and labels larger than a ZFP. Methods of producing solid state nanopores over a wide range of dimensions, e.g., up to 20 nm or greater, using sputtering ion beam techniques have recently been developed. See, U.S. Ser. No. 09/602,650, which is incorporated in its entirety by reference.

Because a nanopore can detect and “read” single molecules, the present invention provides a method for analyzing nucleic acid samples without requiring amplification. Thus, the haplotype of a genomic sample can be directly determined. Although a statistical sampling will be needed to establish a high degree of confidence in the measurement, it is likely that this will require the measurement of no more than 200 target molecules. This corresponds to about 500 picograms of genomic material, e.g., human genomic material, which can be directly obtained using standard sampling methods.

In one or more embodiments of the present invention, double-stranded nucleic acids are analyzed. In one or more embodiments, direct characterization of DNA is contemplated. Information regarding occurrences and locations of specific nucleic acid sequences of genomic DNA (or any other polynucleotide source) is determined according to one or more embodiments of the present invention using sequence-specific binding proteins or oligonucleotides that bind double-stranded DNA. This method is referred to as Protein Binding Encoded Analysis (PBEA).

In one or more embodiments of the present invention, zinc finger proteins (ZFP) are used as labels for haplotyping with a nanopore. Zinc fingers are one of the most common DNA-binding motifs found in eukaryotic transcription factors. Zinc finger proteins typically contain several fingers, each of which is composed of about 30 amino acids. Several of these amino acids in each finger interact in a sequence-specific manner with three adjacent base-pairs of double-stranded DNA, and in some cases RNA (see, e.g., Miller et al., (1985) EMBO J., 4:1609-1614; Wolfe, et al. (2000) Ann. Rev. Biophys. Biomol. Struct. 29:183-212. The modular structure of ZFPs, and the wide variety of sequences they can recognize, have made them an attractive motif around which to design novel DNA-binding proteins for research, diagnostics, and gene therapy (see, e.g., Pabo et al. (2000) J. Mol. Biol. 301:597-624). For the diagnostic purposes described here, using the alternate binding orientation that would make contacts with the purine-rich strand is contemplated. Importantly, ZFPs can be designed to have strong affinities for their cognate DNA binding site, exhibiting high apparent binding constants (K_{d} in the low to sub nanomolar range for wild-type 3-finger ZFPs) with excellent specificity constants (K_{d} non-cognate/K_{d} cognate of about 100 fold or better, see e.g., Paveletich et al. (1991) Science 252:809-817). Far greater affinities and specificities can be achieved with designed ZFPs containing structured linkers or fused dimerization domains that promote cooperative binding of the zinc fingers to their DNA binding sites (see, e.g., Choo et al. (1993) Proc. Natl. Acad. Sci. USA 92:344-348; Elrod-Erickson, et al. (1999) J. Biol. Chem. 274:19281-19285). These properties enable ZFPs to bind DNA samples of high sequence complexity (e.g. human genomic DNA) with high specificity.

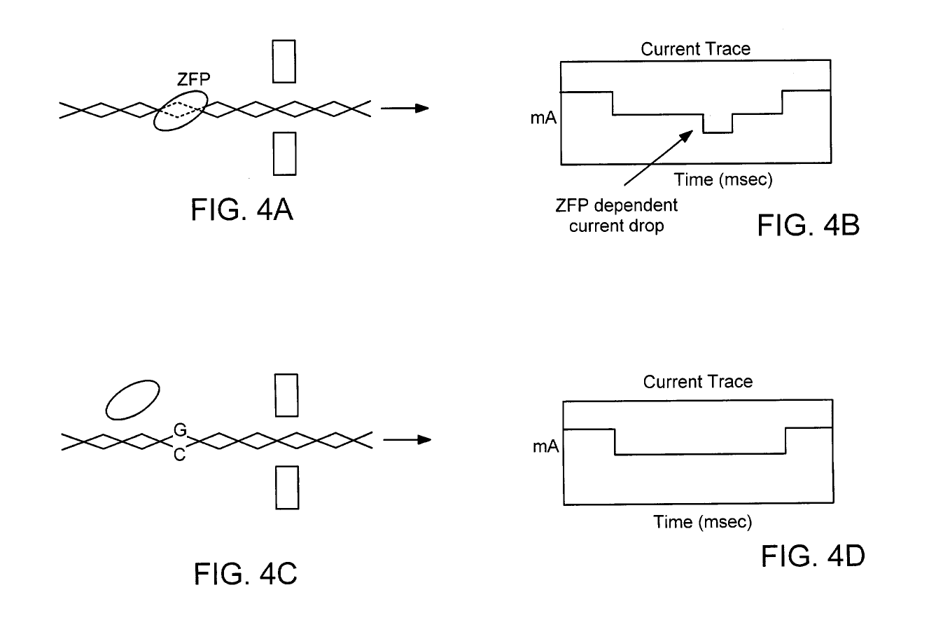

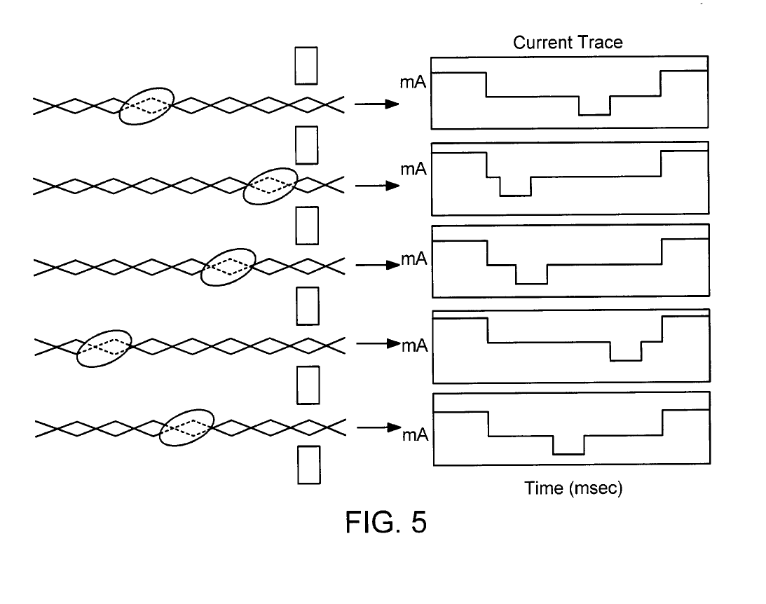

For genotyping in its simplest mode, a Cys₂His₂ zinc finger protein can be chosen to bind a polymorphic site on double-stranded DNA. For example, a DNA fragment in a sample to be interrogated contains the 9-mer sequence CAGAATGCT with the bold A corresponding to an SNP locus (FIG. 4). To this sample, a 3-finger ZFP is added which is designed to bind to the CAGAATGCT site. When an appropriate voltage (trans side of the membrane positive) is applied across the membrane containing a 4-5 nm pore, the ZFP/DNA complex will be drawn through the pore. This will result in an initial drop in the current caused by the DNA alone, followed by an additional drop as the larger cross-sectional ZFP region of the complex translocates through the pore. In contrast, when the same ZFP is added to DNA that contains the CAGAGTGCT allele of the 9-mer sequence (FIG. 3, bottom), no ZFP/DNA complex is formed. This results in only an initial drop in the current due to naked DNA translocating through the pore.

ZFPs can also be used to label invariant (non-polymorphic) sites. Using ZFPs directed to invariant and variant sites, each of many different sequence fragments in a single sample can be distinguished and identified as each translocates through the nanopore. Thus, a single nanopore could genotype a mixture containing multiple different DNA sequence fragments, each of which would contain different SNP loci. Since very long strands of DNA can be translocated through a nanopore, it is possible to use a single nanopore to identify the allele present at multiple different SNP sites along the length of such a strand (haplotyping) since the position along the DNA's length is measured by the position of the current blockade due to the ZFP-DNA complex within the longer blockade due to the entire length of DNA having translocated through the nanopore. Additionally, by combining the concepts outlined above, complex mixtures of different fragments, even random-length fragments, can also be analyzed using this method by using ZFPs, or other labels including, but not limited to, oligonucleotides or chemical labels that permit the identification of each nucleic acid molecule as it translocates through the nanopore. Regions at one or both ends of the DNA fragments are most conveniently allocated for the purpose of identifying the fragments, although other regions could also be used.

To demonstrate how this invention identifies each nucleic acid molecule as it traverses the nanopore, Example 1 focuses again on the description of ZFPs as but one example of the many kinds of label and specific considerations that may be used to identify each of many different DNA fragments as they translocate through the nanopore.

Because the intention is to interrogate many DNA fragments simultaneously, and because in one or more embodiments the region of the genome from which each of the different sequence fragments arises is identified, it is useful to begin by considering the target complexity that can be confidently identified with the least number of probes. Nanopore detection will obviously be limited by the minimum detectable lengths of labeled and unlabeled lengths that can be distinguished. Considerations related to the complexity of the substrate, and the size of the probe to be used, and the selection of single probes as well as libraries of probes are described herein in more detail in Example 1.

In one or more embodiments, the modular nature of ZFPs is used to generate multimeric ZFPs with enhanced target specificity and increased discrimination against single nucleotide changes in DNA. Although a single base change can yield a 100-fold affinity reduction in a 3-finger ZFP, strategies in which multiple ZFPs are covalently linked or linked to peptides that mediate dimerization only upon binding of two ZFPs to adjacent cognate sites provide another means of probing SNPs. Two-finger ZFPs with modest affinities but with dimerization sites that promote cooperative binding upon recognition of cognate DNA sequences are especially attractive, as they reduce the risk of nonspecific bindng that may occur with the equivalent number of fingers linked covalently. These assembled dimers provide more than adequate affinities and specificities for SNP labeling. For example, a fusion protein containing fingers 2 and 3 of Zif 268 fused to the cFos leucine zipper (the cFos leucine zipper provides the potential dimerization site for another leucine zipper) alone does, not bind to its DNA recognition site and an analogous 2-finger protein fused to a c-Jun leucine zipper demonstrates barely detectable binding to its recognition site, but a mixture of the two proteins formed a stable complex with their cognate DNA bindings sites with an apparent dissociation constant of 4.3 nMolar (Pomerantz, J. L., S. A. Wolfe, and C. O. Pabo. (1998) Biochemistry 37:965-970; Wolfe, S. A., E. I. Ramm and C. O. Pabo (2000); Structure 8:739-750; Wang, B. S. and C. O. Pabo (1999) Proc. Nat. Acad. Sci. USA 96: 9568-9573). Their effective specificity renders them sensitive to single base changes in their cognate binding sites, such single base changes causing greater than 100 fold reductions in apparent affinity. The multimerization of individual 2-finger ZFPs also permits the creation of a universal library that can identify up to a 12-base pair sequence (four fingers for up to 4¹² or 1.67×10⁷ different binding sites) from a bank containing only 4⁶, (4,096) different ZFPs fused to dimerization sites.

Multimerization of ZFPs is accomplished by covalently joining the monomeric components, or by creating hybrid proteins of 2 or 3-finger ZFPs fused to moeities that promote dimerization and cooperative assembly after binding to the cognate site on DNA. For example, ZFPs can be fused to the coiled-coil dimerization domain of GAL4, the dimerization domain of various leucine zippers, or random peptide sequences selected from phage display libraries using techniques known in the art (see e.g., Ausubel et al., Current Protocols in Molecular Biology, John Wiley & Sons Inc., New York City, N.Y. 1993). ZFPs can also be modified to be joined to other groups, including, but not limited to, carbohydrate moieties, biotin, streptavidin, and other chemical groups that will promote ZFP dimerization.

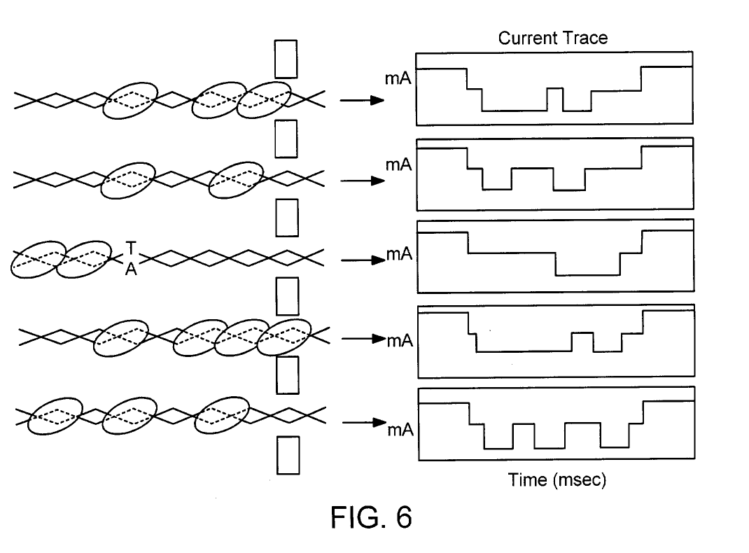

Complex mixtures of random-length fragments or fragments where the SNP site cannot be predetermined can also be analyzed using this method. In this mode, additional ZFPs are selected to specifically bind the fragments at defined locations, which will result in an unambiguous “coded” pattern for each fragment within the sample mixture. See FIG. 6. In this mode, the ZFPs can be chosen to bind at both defined spacings and in contiguous stretches. Importantly, this mode should allow for the analysis of both very long (>100,000 bp) DNA fragments and fragment mixtures with very high sequence complexity. For example, three contiguously bound ZFPs would occupy a binding site of 18 base-pairs which would, on average, be present in only one location within the human genome (4¹⁸=6.9×10¹⁰). Thus, an unambiguous “coded” pattern can be generated for individual chromosomes using this type of strategy. It is also possible that the location of the SNP loci themselves would be sufficient to encode the identity of the fragment while simultaneously revealing the identity of allele at each given loci.

ZFPs satisfy many of the requirements of a successful PBEA system. First, they provide a class of proteins that can be easily engineered and manufactured to bind double stranded DNA in a highly sequence-specific manner. In order to have the 1necessary agent specificity, the protein must be able to discriminate among single-base pair sequences within a defined binding site 6 base-pairs or greater. Second, the affinity of the protein for the DNA (K_{d}) is sufficiently tight to ensure DNA binding using modest concentrations of protein (nanomolar) at the anticipated low concentrations (˜attomolar) of genomic DNA. Third, the t_{1/2} of the protein/DNA complex, which is dictated by the k_{off}, is greater than the time required for the DNA fragment to translocate through the pore. Fourth, the overall shape and size of the protein is such that the difference in the local cross-sectional dimension for the bound and unbound regions of the traversing molecule is reflected in the ionic current signature.

In one or more embodiments of the present invention, the haplotype of a genomic sample is determined. Because the method detects single molecules, there is no need to amplify the target molecules. Thus, a genomic sample can be analyzed directly. Clearly, a statistical sampling will be required in order to establish a high degree of confidence in the measurement. It is likely that this will require the measurement of approximately no more than 100 individual molecules. This corresponds to about 300 picograms of human genomic material, which can be easily obtained using standard sampling methods. Moreover, the slow off rate (k_{off}=100 minutes) of a ZFP for its cognate binding site and relatively high rate of translocation of the DNA through the pore (˜>1,000 base-pairs per second) provides the opportunity to detect the connectivity (haplotype) of two loci located as far apart as ˜one million base-pairs, assuming the ability to maintain fragments of sufficient length during the sample preparation and analysis process.

An additional advantage of the analyses described above, is that they can be performed using universal library of ˜4,096 2-finger ZFPs. This number is actually likely to be less since there is some latitude with regard to the selection of a ZFP for a defined 6-mer site. Clearly, this attribute eliminates the need for generating the large number sequence-specific reagents (>one million) for each SNP that would need to be analyzed (although this is contemplated as within the scope of the invention). ZFPs can be engineered to be sensitive to the methylation state of the DNA. ZFPs of this sort will have utility in DNA analysis.

In some other embodiments of the present invention, short oligonucleotides are hybridized to discrete regions along the single stranded RNA or DNA. This method is referred to as Oligonucleotide Hybridization Encoded Analysis (OHEA) and is illustrated in FIG. 2. A nanopore 20 is provided in a substrate 22. In this example, the channel defined by the pore serves as a limiting or defined volume for the translocation of the target nucleotide. The constraining dimension of the pore, that is, its narrowest aperture, serves as the detector. Oligonucleotides 24, 26 hybridize selectively to complementary regions of a single stranded nucleic acid strand 28. A bias is applied across the substrate 22 to drive the hybridized nucleic acid through the pore 20. The greater cross-sectional bulk of the hybridized regions yield a distinct signal as the target molecules traverses a pore. Distinctions may be made between different hybrids based upon the change in signal amplitude and the duration of the change.

FIG. 3 illustrates this principle. Three oligonucleotides are added to a solution containing a single stranded DNA to be analyzed. The oligonucleotides hybridize to three different regions of the target single strand nucleic acid, and the hybrid complex traverses the nanopore (FIG. 3A). The resultant current signal is shown in FIG. 3B. The signal drops initially as the polymer enters the pore. The signal is reduced even further when the more bulky hybridized regions enter the pore. Based upon the location of the reduced current signal relative to onset of initial current drop, the location of the hybridized regions on the DNA sample can be identified. In addition, identification of the oligonucleotide, e.g., by length or other unique identifier, permits determination of the hybridzation sequence.

Multiple sites of the molecule can be probed simultaneously. OHEA retains the connectivity among the segments since it does not necessitate the separation of the genetic material into fragments. The ability of OHEA to incorporate the added connectivity information makes it much more powerful than traditional methods. There is no known limit to the length of the translocating polymer in this system. For example, 1,300 base-long homopolymer can be routinely translocated through an α-hemolysin channel with consistent and predictable electrical behaviors while a 35,000 base-long polynucleotides have also been detected as illustrated in figure X below. Thus, OHEA can be used to probe a large number of sites on a single continuous stretch of DNA. In at least some embodiments of the invention, an oligonucleotide is used which binds to a specific sequence found at multiple sites on a single continuous stretch of DNA. In at least some embodiments of the invention, a mixture of oligonucleotides is used which includes multiple oligonucleotides which bind to different specific sequences found at different sites on a single continuous stretch of DNA.

OHEA can utilize mixtures of either genome specific, allele specific, or other sets of universal oligonucleotides. The genome-specific and allele-specific approaches are both agent specific approaches, which are designed to distinguish sequences associated with a particular agent, i.e., the genetic material associated with a particular individual, species of organism, pathogen, allele, or other unique sequence from among a set of other sequences. For the agent-specific approach, the goal is to define a relatively small set of oligonucleotides that will encode a defined genetic material in such a way as to unambiguously distinguish it among a defined set of predetermined agents. For example, the oligonucleotide encoding target site (k) may be limited for any given agent's genome to an arbitrarily defined 10,000 nucleotide region. It may also be assumed that each coding segment (λ) within the target site k can be between 12 and 25 nucleotides in length (1 in FIG. 2) and located anywhere within a defined 100 nucleotide region of the target site. This will give a defined number of encoding windows equal to k/λ or in this case, 100. If the number of encoding oligos (r) which can be assigned to the 100 available windows is limited to 5, then the theoretical number of distinguishable patterns that could be generated for any given agent is equal to (k/X)!/r! (k/λ−r)! or approximately 10⁸ for this example. Importantly, this calculation assumes that the nanopore measurement can resolve single 100 nucleotide windows along the entire 10,000 nucleotide target region. In other words, the measurement distinguishes between a duplex at window 99 from a duplex at window 100. This corresponds to a resolution of approximately 100 in 10,000 or 1%.

For the universal OHEA approach, a universal set of oligonucleotides are provided that will encode an ion current signature for any given agent's genetic material which will be distinguishable from all other possible agents' signatures at some defined statistical confidence level. This approach will be analogous to traditional methods where an unknown agents' genetic material is cleaved by a defined restriction endonuclease and the resulting fragment pattern is then compared with that of a known database. In the present case, the encoding oligonucleotide mixture is analogous to the restriction sites and will be defined based on theoretical simulations; however, the method provides the additional advantage that the connectivity of the sample nucleotide is not lost.

For both the agent-specific and universal approaches, the length of the duplex region along the nucleic acid molecule can be varied to achieve a greater distinction among different nucleic acids. This can be accomplished by varying either the length of a single encoding oligonucleotide or using multiple oligonucleotides that hybridize directly adjacent to one another. Understanding both the length and spacing (multiples of k) limits of the system will provide optimal resolution. Increasing the total number of oligonucleotides in an agent-specific encoding mixture will increase the resolution and thus enable greater distinction among more subtle variants of a given agent species.

It is also apparent that multiple nucleic acid molecules can be analyzed simultaneously using this method. For example, the molecules to be analyzed can be such that the sequence sites of interest are located at predetermined distances from the nucleic acid termini. The identity of each molecule in the mixture can be determined by time at which the current drop occurs as the molecules traverse the nanopore.

In another aspect of the invention, the modification of the defined local area is made directly to the nucleic acid molecule which is to be characterized. The modification is such that the local cross sectional area of the modified polynucleotide can translocate through the channel's limiting aperture, yet is large enough to produce a readily detected current blockage distinguishable from that caused by the unmodified polynucleotide. Such modifications include succinimidyl esters, iodoacetamides and maleimides that can be covalently linked to individual nucleic acids of the polynucleotides. RNA or DNA molecules are converted into distinctly modified DNA by using primers modified with differently spaced bulky molecules. Since the modifications on the primers are known, DNA containing the expected current blockage patterns can be distinguished in a given mixture. The primers that were not extended can be distinguished from the reverse transcriptase or polymerase products because the transcripts are expected to be significantly longer. Thus, unextended primers need not be separated from the mixture before conducting the nanopore translocation assay. The capacity to distinguish a wide range of DNAs or RNAs in a single mixture is determined by the number of differently modified probes that can be resolved by the nanopore.

The reverse transcription with modified primers may be optimized using standard methods on test templates with predictable product lengths. In one or more embodiments, avian myeloblastosis virus (AMV) reverse transcriptase is used in generating full-length transcripts. If 12 bases represent the minimal spacing needed for resolution on the modified primers, 3 distinct modifications, one at the 5′ end, can be made in the space of 24 bases. Combinations of the presence and absence of bulky groups at these positions will yield 8 different blocked current patterns. It is conceivable that placing two bulky groups close to each other can expand the number of different codes by introducing prolonged current dips, and longer primers will allow for more distinct patterns. Additionally, abasic segments and other molecules with increasing or decreasing bulk, including, but not limited to naphthofluorescein and dansyl derivatives, may induce different levels of current changes. Most of these molecules are commercially available as phosphoramidites or in amine or thiol reactive forms that can be readily conjugated to the oligonucleotide primers. Any of a large number of labels or modifications can be used. Furthermore, the modifications may interact with the channel so as to prolong translocation duration as well as causing an additional blockage to current flow through the nanopore. If the modifications to the nucleotides are to be introduced into the polymer by reverse transcriptase or polymerase, they must obviously be selected so that they do not interfere with the reverse transcriptase or polymerase reaction.

Additionally, this method can be applied to modify primer extension assays, particularly for quantitative comparisons between transcripts of extremely divergent lengths. Chemically modified primers can be designed to produce similar length transcripts in primer extension assays, including, but not limited to nuclear runoff assays, making this class of traditional molecular biology technique an absolute quantitative process.

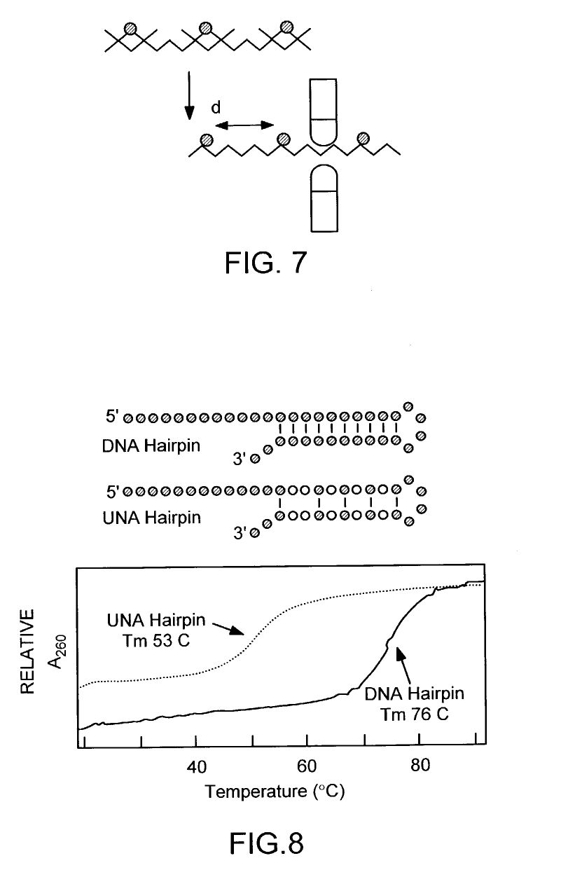

In one or more embodiments of the invention, a universal oligonucleotide ligation method (UOLA) is employed to characterize the target nucleic acid molecule. In this method, a mixture of discrete short X-mers (e.g., 6-mers) is fabricated such that each X-mer is associated with one of 10 different discrete tags. A tag may be some type of chemical moiety that is covalently attached to the X-mer, which will generate a unique and identifiable ion current signature in the nanopore. Alternatively, the tag could be “encoded” into the inherent length of X-mer itself using varying numbers of universal nucleotides such as 5-nitroindole (Z). For example, the amount of information content within the 6-mer sequence AGACTG is equal to that of the 9-mer AGAZZZCTG and 12-mer AGAZZZZZZCTG. These X-mer mixtures are then hybridized with a single-stranded nucleic acid molecule and treated with a DNA ligase. This will result in the ligation of those X-mers that coincidentally hybridize directly adjacent to one another. The resulting ligated products will then be stripped away from the target and analyzed using the nanopore. See FIG. 7.

In one or more embodiments of the present invention, the UOLA method is used to identify pathogens in a test sample. In one or more embodiments of the present invention, sets of random sequences are provided that represent various bacterial genomes of the pathogen to be detected. Contacting a single-stranded test sample with the sequence sets under hybridizing conditions, followed by ligation, results in ligated products unique to the pathogens in the test sample. Using sets of random sequences to represent various bacterial genomes, the ligation products using a X-mer mixture comprising only 70 unique X-mer sequences having the information content of 6-mers, each tagged with one of 10 discrete tags, can distinguish among approximately 90 arbitrarily chosen. Importantly, by increasing the number of discrete tags, and hence decreased ambiguity between the X-mer sequences and the tags, the number of genomes that can be distinguished increases.

In another aspect of the invention, a method is provided that significantly improves Restriction Fragment Length Polymorphism (RFLP) analysis—a well-established approach for genetic typing bacteria, virus and bacteriophages. In the conventional method, double-stranded chromosomal or plasmid DNA is isolated and cut with one or more defined restriction endonucleases that recognize anywhere between 4 and 8 defined base-pairs. The dsDNA fragments are then separated by length using gel electrophoresis and visualized by either isotopic fluorescent-dye labeling.

The resulting number of restriction fragments can be quite large, giving very complex gel band-patterns. For example, the number of restriction sites (r) that have a 6-base pair recognition site within a target length (L) will be equal to L/4⁶ or r=L/4,096. Thus the average number of restriction sites for an organism having a genome of 3×10⁶ base-pairs will be approximately 3×10⁶/4,096 or 730.

At least two mechanisms can give rise to a fragment length difference among two closely related samples. First, as little as a single base-pair difference between the two samples can either add or remove a restriction site and hence shorten or lengthen the fragment associated with the site in question. Second, an insertion or deletion anywhere along the genome can result in the shortening or lengthening of a fragment that encompasses the insertion or deletion. Importantly, both types of differences can be difficult to detect with current gel-based methods. Because RFLP analyses results in 100s to 1000s of discrete fragments, there will be multiple fragments of similar size. These will not be well resolved by the gel electrophoresis, making it difficult if not impossible to unambiguously assign and quantify each discrete fragment in the mixture.

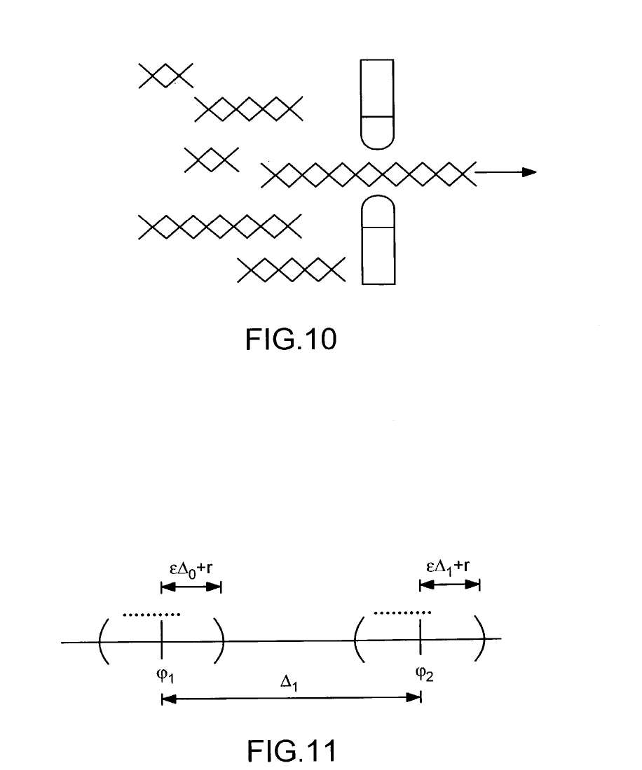

In one aspect of the present invention, fragment length analysis using nanopore technology (FIG. 10) is used to analyze a fragment mixture in order to determine fragment lengths in the population. The method of the invention provides information for a fragment mixture having a much broader range of lengths than can a standard gel-based method. For example, there is a linear relationship between the log of the electrophoretic mobility of dsDNA (μ), and the gel concentration. Importantly, this relationship holds true for only about one log in μ, or about a factor of 10 in DNA length when the migration distance is about ˜10 cm or greater. Furthermore, because this relationship also tends to deviate from linearity at short migration distances (˜<0.5 cm), it is generally necessary to perform the measurement at more than one gel concentration in order to accurately resolve all of the fragments in the mixture when the length distribution of that mixture is greater than a factor of 10. Thus, having the ability to size a broader range of fragments at a defined level of resolution under a single defined assay condition should allow for a more powerful analysis of the mixture without increasing the analysis time.

Gel based RFLP is generally not considered to be sufficiently quantitative to discriminate between two sample mixtures whose fragment patterns differ only by the number of fragments of a given similar length. However, at relatively high DNA concentrations (˜μM), counts of 200 molecules/minute through a nanopore can be easily attained and a nanopore-based RFLP could be used for this purpose. In a reduced complexity mixture containing only 10 different types of fragments, (which could be generated by using either agent-specific PCR, AP-PCR or RAPD), one would expect to determine the relative concentration of each fragment to a precision of roughly 5% (400^{1/2}/400≅0.05) within a 20 minute assay period. Complete translocation of the all molecules in the cis chamber is not necessary as long as statistically significant numbers of molecules are measured.

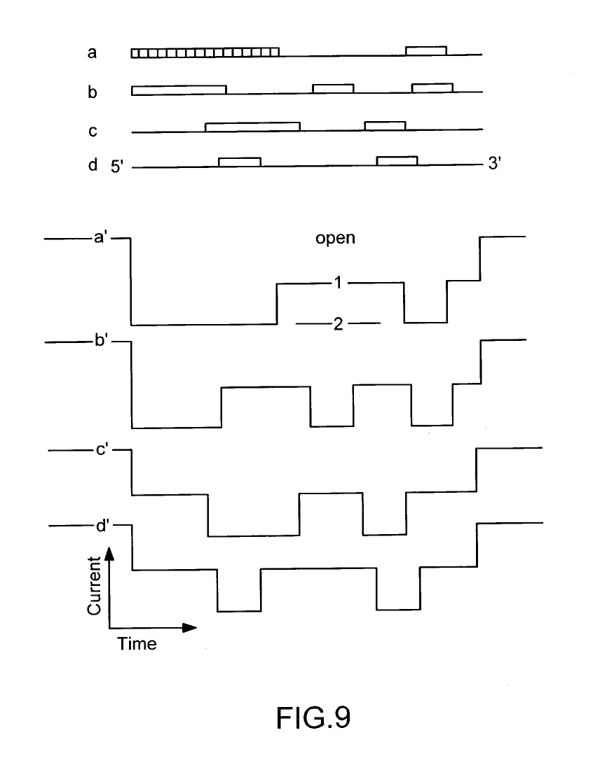

A solid-state nanopore provides the ability to probe for particular RNA or DNA analyte types in a mixture of many RNA or DNA types is enhanced. Because the diameter of a solid-state nanopore can be selected, the size limitations for modifying the target RNA or DNA molecules so as to make them readily distinguishable during translocation in the nanopore are less restrictive. Although assays and sample preparations similar to those conducted with a protein channel can be performed, the analysis of an RNA analyte using a solid-state pore can, for example, be a direct measurement, without any transcription. By eliminating the steps needed for transcription, analyte detection is simplified and quantification error, caused by transcription bias among different types, is eliminated. The preferred preparatory step is simply to anneal, to each of the analytes of interest, appropriate segments of probes. This may be done, in one or more embodiments, by adding many probes to a mixture of full length RNA derived from a tissue or cell sample. This is a method for mRNA detection without amplification and with minimal, if any, RNA segmentation. Instead of using oligonucleotides as primers to enzymatic reactions or probes in Northern blots or Ribonuclease Protection (RNP) assays, these oligonucleotides are used as markers or labels for target mRNAs molecules or even specific exons within each mRNA (FIG. 9). Hybridization between a marker oligonucleotide and the target mRNA creates a double-stranded segment in an otherwise single-stranded messenger region. When necessary, the oligonucleotide probes are designed and placed so as to deter native intra-molecular base pairing that can interfere with polynucleotide translocation.

A large number of molecules can be assayed simultaneously with very small samples. By carefully selecting different oligonucleotides probes, the number and length of duplex regions and the spacing between them along the linear mRNA can be varied and controlled on the basis of available sequence information to maximize the number of different mRNA and/or exons one can detect in a sample. Neither isolation of the target molecules from other polynucleotides nor removal of excess oligonucleotide probes will be necessary because only the target molecules will have the distinct signal of alternating single-stranded region and duplex segments. When there is no polymer in the nanopore, the magnitude of the current through the pore is equivalent to the open pore current (FIG. 9, “open”). When a single stranded region of the polymer is translocating through the nanopore, the current drops to a partially blocked state (FIG. 9,“1”), whereas during translocation of heterduplex regions, the current is further diminished (FIG. 9, “2”).

Since we are not aware of length limits on the translocating polymer in our system (we have driven 35,000 base-long oligonucleotides through the α-hemolysin channel), potentially the entire layout of full-length mRNAs or entire genes can be examined. Strategic design and placement of the oligonucleotide probes at critical positions can yield information on the chromosomal phasing or haplotype of two or more nucleotide polymorphisims or the relative population of alternatively spliced, truncated, and rearranged messages. Thus, DNAs or mRNAs with different sequences and mRNAs with alternate splicing can be detected, distinguished, and counted in a given mixture. Furthermore, separate analysis of nuclear and cytoplasmic mRNA will provide information on mRNA processing, furthering our understanding of mRNA transport, decay, and expression in cells under different conditions.

To ensure complete hybridization of target DNA or RNA segments, it may be necessary to completely denature the mRNA followed by hybridization with excess probes. RNAs can be effectively denatured with heat and dimethyl sulfoxide with no detectable breakdown. In one or more embodiments, modified oligodeoxynucleotide probes containing 2-aminoadenine, 2-thiothymine, C-5 propynyl-dC, C-5 propynyl-dUand other 2′-modified oligonucleotides, including “LNAs” (Locked Nucleic Acids) can be used, all of which have been shown to increase thermal stability with their complementary sequences. As a result of the tighter binding between the modified and natural oligonucleotides, these heteroduplex regions, once formed, will discourage any undesired intra-molecular base-pairing or random association of the target RNAs. Thus, hybridization schemes can be devised to disrupt sequences of particularly favorable or probable secondary structures. Optimal hybridization conditions will provide maximum hybridization efficiency. “Missed” hybridization sites within a single molecule or a few molecules will be detected and accounted for during analysis if a statistically significant numbers of these molecules are examined. Because molecules are examined individually, if one out of six target duplex sites remain single-stranded on one or a few molecules of a given type, the missed sites will be apparent, and can be discounted as an “error” in the assay when the lengths and current-blockage patterns are compared for the entire sample.

The precision in determining the relative number of molecules of one polynucleotide type vs. another polynucleotide type will be statistically limited by bias between molecules and by the number of translocation events that are counted. Bias between molecules can be evaluated using test samples of the target polynucleotides to determine if, and to what extent, the probability of a target polynucleotide “finding” the nanopore and translocation through the nanopore is affected by molecular weight, hybridization pattern, terminal charge state, etc. To assure that an adequate number of translocation events are counted, the sample should be as concentrated as possible and the assay should proceed for as long as is required to assure the desired precision.

The precision with which the relative concentration of any two or more different polynucleotides is determined will obviously be limited by the number of molecules that are counted. Depending on the concentration of molecules near the nanopore, counts of 200 molecules/minute are easily attained. The counting precision will be no better than the standard deviation of the number of polynucleotides that are translocated divided by that number. Thus, in a sample containing 10 different mRNAs at roughly equal concentrations, one could expect to determine the relative concentration of each to a precision of roughly 5% within a 20 minute assay period (200 molecules/minute×20 min/10 molecule types)^{1/2}/(200 molecules/minute×20 min/10 molecule types≅0.05). Complete translocation of the entire cis chamber is not necessary as long as statistically significant numbers of molecules are collected.

In comparison to a gene chip, which can examine the relative expression levels of tens of thousands of messages simultaneously within 24 hours, the technology described here has other distinct advantages. It has the potential to yield absolute quantitative information on the target DNAs or mRNAs; it is not subject to biases due to enzymatic amplifications, and moderate sample-to-sample variations in probe quantities will not affect counting results. In contrast to the gene chip, which is limited to evaluating segments of interest, our technology can map long segments of polynucleotides or mRNAs at high resolution, thus providing valuable information about DNA haplotype or mRNA processing during different cellular states. The ability to simultaneously detect localized proteins on multiple regions of nucleic acids at single molecule resolution will provide insight to important problems in nucleic acid processing that involves nucleic acid-protein interactions. A few examples are viral gene processing, RNA splicing, and nonsense mediated decay.

Application of this technology will include, but will not be limited to the following:

1. Assay of relative or absolute gene expression levels as indicated by mRNA, rRNA, and tRNA. This includes natural, mutated, and pathogenic nucleic acids.

2. Assay of allelic expressions.

3. Haplotype assays and phasing of multiple SNPs within chromosomes.

4. Assay of DNA methylation state.

5. Assay of mRNA alternate splicing and level of splice variants.

6. Assay of RNA transport.

7. Assay of protein-nucleic acid complexes in mRNA, rRNA, and DNA.

8. Assay of the presence of microbe or viral content in food and environmental samples via DNA, rRNA, or mRNA.

9. Identification of microbe or viral content in food and environmental samples via DNA, rRNA, or mRNA.

10. Identification of pathologies via DNA, rRNA, or mRNA in plants, human, microbes, and animals.

11. Assay of nucleic acids in medical diagnosis.

12. Quantitative nuclear run off assays.

13. Assay of gene rearrangements at DNA and RNA levels, including, but not limited to those found in immune responses.

14. Assay of gene transfer in microbes, viruses and mitochondria.

15. Assay of genetic evolution.

16. Forensic assays.

The following examples are described which illustrate the invention. They are not intended to be limiting of the invention.

### EXAMPLE 1

### Statistical Considerations and Selection of ZFP Probes

Assume that the technology can distinguish an unlabeled DNA stretch of length X from an adjacent labeled stretch of length λ. That is, a fully labeled DNA stretch of length 2λ can be distinguished from a 2λ length of DNA in which the labeled region is only λ long and the unlabeled length is λ. Furthermore, assume that the analysis used to derive information from the assay relies on a k long region of each sequence fragment to be interrogated, where k represents the number of DNA “windows” of length λ, none of which should be known polymorphic sites. We then assume that the 5′ edge of each window in a given DNA target sequence is the potential starting point for binding of a ZFP label. Using the appropriate probes of length ≦λ, we envisage a binary coding scheme where at each window in the grid (λ₁, λ₂, λ₃, . . . λₖ) we either have a ZFP probe that binds at that window, or not. This binary coding scheme can distinguish 2^{(k/λ)} sequences, using specific probes of length ≦λ. The binary code assigns a word wξ{0,1}^{(k/λ)} to every target DNA sequence. A “1” in the ith position in w is represented by a ZFP label at the ith window (λᵢ) of the sequence.

For a specific example, let k=1500 bp and λ=50 nucleotide pairs, much more than enough space in which to bind a 3 finger ZFP or even a femtomolar affinity, very high specificity 6-finger ZFP that would extend over 18 contiguous base-pairs (Pabo et al. (2000) J. Mol. Biol. 301: 597-624; Moore et al. (2001) Proc. Natl. Acac. Sci. USA 98:1432-1436). Based on results with ssDNA translocating through α-hemolysin, 100 nucleotide pairs (100 bp=2λ in this example) is a very reasonable length within which one is able to resolve and distinguish labeled from unlabeled DNA in a nanopore. Using these figures, k/λ=1500/50=30, we can generate 2³⁰≈10⁹ distinguishable patterns. This is of course much more than necessary for any practical purpose, and simply illustrates that message target complexity is not a concern. What is of concern is that we would normally want to limit the number of probes needed to generate a unique characteristic pattern for every different DNA target sequence fragment. Using a binary coding scheme as above, but constraining the number of probes to p per sequence, we can design (k/λ)!/p! (k/λ−p)! distinguishable patterns. Limiting ourselves to p=4 with the parameters discussed above, we then still get more than 27,000 different patterns, more than enough for the entire human transcriptome broken up into 150,000 bp or longer sequence fragments (since 3,000,000,000/150,000=20,000<27,000). With p=2 only, one can still identify about 400 target sequence fragments, needing only about 800 synthesized ZFP probes, 2 for each sequence fragment.

The above considerations assume that a set of ZFP labels separate from those required for detecting SNPs and haplotyping will be required either to identify the sequence fragments or to determine the directionality of the translocating fragment. While this may prove to be the case in many determinations, it is also possible, as discussed below, that the same ZFP labels used for detecting SNPs and for haplotyping will themselves suffice, or at least help, to identify the sequence fragments and their directionality.

As we consider haplotyping and ask how many different SNP loci can be probed by a single nanopore in a single assay, it becomes clear that the type of ZFP used (2-finger, 3 finger, etc.) will materially affect the number of SNPs that can be probed per assay. Furthermore, we will see below that a key factor is the precision with which the duration of a translocation event signal can be equated to the actual number of base-pairs that have been translocated.

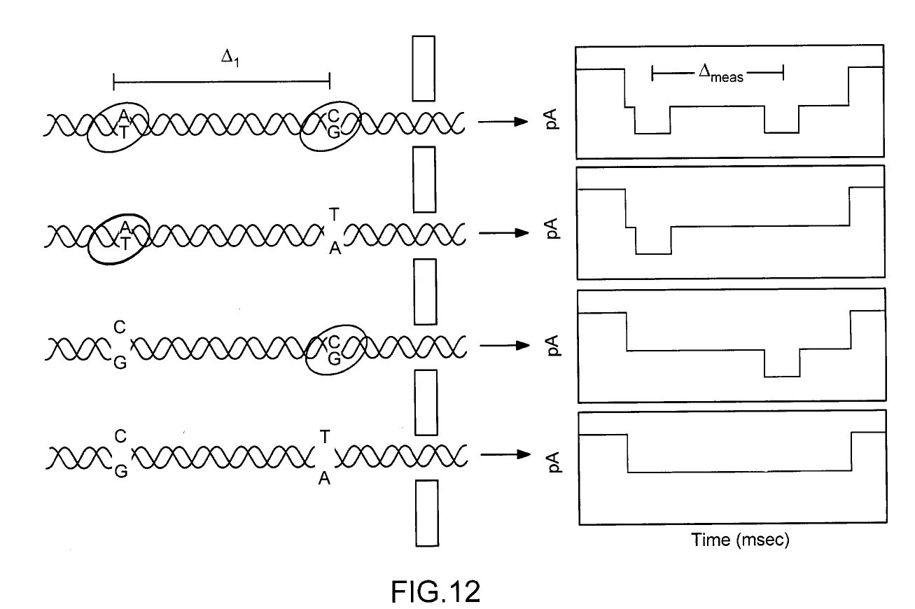

Assume a DNA sequence fragment of indefinite length with s SNP loci (Φ₁, Φ₂, . . . , Φₙ. We select s different ZFPs, w₁, w₂, w₃ . . . , wₛ, such that each of these ZFPs binds specifically to only one of the alleles of the ith SNP, but to no other SNP. As a result each SNP locus will either be labeled with a ZFP (if the specific allele appears) or will remain unlabeled (if the other allele appears at this polymorphic site). An example of such a scenario is shown in FIG. 11. A sequence fragment (solid horizontal line) containing 2 SNPs (two vertical lines), Φ₁ and Φ₂, separated by distance Δ_{I}. Specific ZFPs (dotted lines), one designed to bind to one of the alleles of Φ₁, the other selected to bind to one of the alleles of Φ₂, can label (or not label) each of the SNP loci. The actual physical distance between Φ₁ and Φ₂ in base pairs is designated as Δ_{I}. The measured distance between the position of Φ₁ and the position of Φ₂ is subject to error whose limits are designated by brackets on the sequence fragment. Taking for example a short sequence fragment containing only two SNPs (that is, where s=2 as in FIG. 11), a nanopore will make it possible to distinguish between 2^{s} possible configurations corresponding, in this case, to all four possible s-fold haplotypes (FIG. 12; note that only the labeling at polymorphic sites is shown; labeling that would identify the sequence fragment and its directionality during translocation is not shown). As shown in the right hand portion of FIG. 12, reading the lengths of time between different current blockades will be critical if a nanopore is to distinguish between different haplotypes.

Since the nanopore length reading errors are a combination of an additive error, r, in units of base pairs, and a multiplicative error ε, that is a fraction of the distance, Δ_{I} in base pairs, between the ith SNP and the (i+1) SNP that are to be probed, the true physical distance, Δᵢ, between two SNPs will be measured as a distance Δₘₑₐₛ (FIG. 12) that lies somewhere between (1−ε)Δᵢ−r and (1+ε)Δᵢ₊₁+r (that is, Δ(1−ε)Δᵢ−r≦Δₘₑₐₛ≦(1+ε)Δᵢ₊₁+r). To avoid errors that would lead to incorrect haplotyping, a “clean region” of length=ε—ᵢ+r on each side of each SNP locus is needed. Clean here means that no other labeling probe used in the assay binds in this region.

In formal language, we can state that a set of s SNPs is collision free if the two following conditions hold:

wᵢ binds to one of the alleles of the ith SNP (but not to the other allele).

wᵢ does not bind to the clean regions of any of the other SNPs in the assay.

Note that there is no requirement about ZFPs binding to regions of the sequence that lie outside of the clean regions of any of the SNPs. On the contrary, it is advantageous if several ZFPs, in addition to binding to their cognate alleles at their designated SNP loci, also bind to invariant sequences that lie outside of all clean regions. Whether they bind outside of all clean regions because of multiple specificities or because of redundant sequences in the fragments that happen to duplicate the SNP being probed, the pattern of SNPs that bind to invariant sequences outside of the clean regions will be predictable based on the known sequence of the fragments being probed. Observing this predicted pattern outside of the clean regions will therefore serve to identify the sequence fragment and its translocation orientation in a mixture of different fragments, and will also help to “recalibrate” the length of polymer that has translocated through the nanopore. This will correct accumulating length reading errors during translocation of very long polymers, or translocation of regions of very widely separated labeled SNPs.

To understand the number of loci that can be jointly interrogated by the proposed nanopore methods, stochastic modeling has been performed. Such modeling and computer simulations assume that the distances between adjacent SNPs are geometrically distributed with mean=500 bps. The simulations draw DNA stretches around hypothetical SNP sites, continuing while the two conditions above can be satisfied by a particular set of selected ZFPs. In other words, each SNP to be identified must have at least one ZFP that binds to one, and only one, of its alleles and this ZFP must not interfere with the clean region of any other SNP.

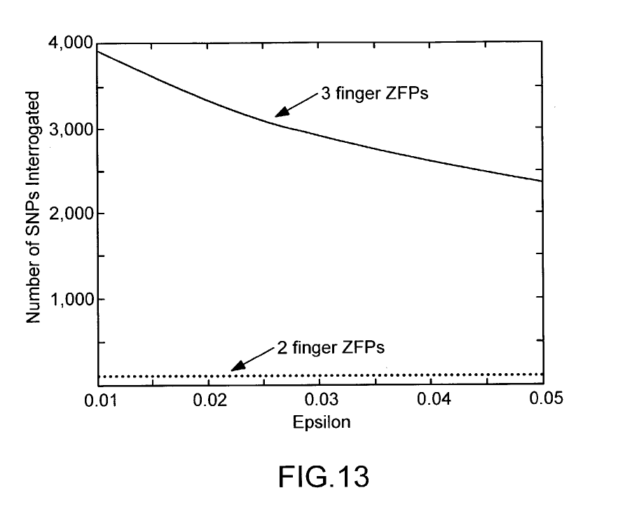

Running this procedure a large number of times the total number of loci that can be interrogated in a collision free manner in one assay with a selected set of ZFPs is estimated for different ZFPs. As an example, FIG. 13 shows the average number of loci that can be jointly interrogated using 2-finger and 3-finger ZFPs, assuming varying resolution parameter (ε) values. Thus, with 3-finger ZFPs, it will be possible to interrogate about 3,900 SNPs in an assay. Assuming that we are interested in s-fold haplotyping, as described above, we divide this number by s, the total number of SNPs to be probed on all of the different sequence fragments, to determine the approximate maximum number of DNA sequence fragments whose haplotype can be determined per assay. If each of the different sequence fragments in the assay is about 30,000 bp long and each contains 30 SNPs recognized by the ZFPs that are used, one should be able to assay about 130 different sequence fragments (3,900/30=130) in one assay using 3-finger ZFPs. Assuming the nanopore assays about 3 fragments per second (translocation duration for a 30,000 pb fragment is only about 60 milliseconds—most of the time is spent waiting for the next polymer end to be captured in the nanopore) and that to assure precision, 100 copies of each fragment is to be interrogated, the entire assay would required only 1.2 hrs (130×100/3=4,333 sec=1.2 hrs) to haplotype the 130 different 30 kpb regions of a chromosome.

While FIG. 13 informs us that a single assay with 3-finger ZFPs can probe 40 fold more SNPs than a single assay with 2 finger ZFPs, there may be reasons to prefer running assays with 2-finger ZFPs rather than 3-fingers ZFPs. Since a 2-finger ZFP 6 will recognize a six base-pair sequence and there are only 4⁵=4,096 possible different 6-base long sequences, a universal library of 2-finger ZFPs could in principle be generated from only 4,096 different ZFPs, whereas a universal library of 3-fingers ZFPs would, using the same reasoning, require 4⁹=262,144 different ZFPs. Thus as many as 262,144 different 3-finger ZFPs would be required to probe a very large number of different polymorphic sites in a few assays whereas the same number of sites could be probed using only 4,096 different 2-finger ZFPs, but at least 64 times as many separate assays would be required. Clearly, the decisions about what kind of ZFP to use would have to take into consideration a number of factors in addition to the availability of ZFPs with the requisite specificity and selectivity. For example, if one needed to establish the linkage between over 100 SNPs along the length of a single chromosome, it would be necessary to use 3 finger ZFPs even if this entailed production and characterization of many novel zinc finger proteins. On the other hand, if one wished to establish the linkage between just 10 SNPs on each of several thousand different sequence fragments from different regions of the genome, it might be easier to process the fragments in batches, performing multiple assays but using an available universal library of 2-finger ZFPs (if 2 finger ZFPs with adequate affinity can be generated).

Distinguishing the ZFP-DNA complex from the DNA alone as it translocates through the nanopore may, in cases where the ZFP is very short, require that an additional label or “tail” be added to the pure XFP protein. A 3-finger ZFP would extend over only 9 bases, thus giving rise to a signal whose duration could be approximately 30 μsec long. Clearly readable signals shorter than 30 μsec have been discerned and measured, but doing so in the context of making continuous measurements during the translocation of a long DNA molecule may be facilitated by extending the signal length. This can be done using standard molecular biology manipulations that will be known to those familiar with the art by engineering a structured polypeptide “tail” into the ZFP construct. This tail should be able to lie against the DNA as it is dragged through the nanopore by the ZFP that is bound to the translocating DNA. Such a tail could be single or multiple repeats of the non-DNA binding finger 4 of the TFIIIA Xenopus transcription factor, or finger 2 of the Zif268 murine transcription factor with serine substituted for the wild type DNA binding amino acids at positions −1, 2, 3, and 6 (Moore et al., supra). Alternatively, should polydactyl ZFPs with greater length and affinity be needed, these amino acid sequences could also be used as linkers that covalently associate two or more 2-finger units to form a ZFP which extends over more than 6 base-pairs. Such strings of 2-finger units, joined by polypeptides that are longer than the canonical linker sequences, exhibit greater specificity and discrimination against single base changes than do the equivalent number of fingers linked by the native or canonical -TGEKP- sequence.

### EXAMPLE 2

### Oligonucleotide Hybridization Encoded Analysis (OHEA)

For OHEA, defined encoding oligonucleotides will be synthesized using standard methods (Caruthers M. et al., Methods in Enzymology, 154; 287-313 (1987)) with sequences defined the particular applications as outlined above. These encoding oligonucleotides may contain duplex stabilizing modifications such as 2-aminoadenine, 2-thiothymine, C-5 propynyldC, and C-5 propynyl-dU, a minor groove binding moiety (MGB) (Nucleic Acids Research 25: 3718-3723, 1997) or Locked Nucleic Acids (LNAs) modifications (Wengel J. et al., (1999) Nucleosides and Nucleotides, 18 1365-1370, Kvaemo L. and Wengel, J., (1999) Chem. Commun., 657-658).

The nucleic acid target material is single-stranded RNA or DNA. The single stranded target is generated using one of a number of methods known in the art such as; assymetric PCR, standard PCR followed by digestion of one strand with exonuclease, or in vitro transcription of RNA targets using a phage RNA polymerase. It is preferred that the single stranded target have little or no intramolecular structures (secondary structure). This can be accomplished using the UNA technology described in Example 1.

The oligonucleotide encoded target molecules are generated by incubating the target material with the defined oligonucleotides under buffer conditions with a pH between 4.5 to 9.5, more usually in the range of about 5.5 to 8.5, and preferably in the range of about 6 to 8. Various buffers that are well known in the art are used to achieve the desired pH and maintain the pH during the determination. Illustrative buffers include borate, phosphate, carbonate, Tris, HEPES, barbital and the like. The particular buffer employed is not critical to this invention but in individual methods one buffer may be preferred over another. The reaction is conducted for a time sufficient to produce the complete or near complete duplex formation.

The concentrations of the encoding oligonucleotides and target(s) vary depending upon the exact application. It is anticipated that the number of single-stranded target molecules can be as low as a single molecule within a sample mixture but generally may vary from about 10² to 10¹⁴, more usually from about 10¹⁴ to 10¹³ molecules in a sample, and preferably at least 10⁶. The concentration of encoding oligonucleotide can be equimolar to that target but usually about 10 to 10² times more concentrated, and preferably about 10³ to 10⁶ times more concentrated. The limiting oligonucleotide concentration will be dictated by that which is necessary to drive stable duplex formation (t_{1/2} must be greater than the target molecule translocation time) under target-limiting conditions at the desired assay temperature.

The ionic current signature for the duplex encoded target molecules will then be analyzed using a synthetic nanopore having the appropriate pore diameter fixed within an apparatus like that previously described (U.S. Pat. No. 5,795,782 & U.S. Pat. No. 6,015,714). The sample may be concentrated at the pore entrance using the electrophoretic concentration method described above.

### EXAMPLE 3

### Protein Binding Encoded Analysis (PBEA)

For PBEA, defined Zinc Finger Proteins (ZFPs) are selected from a library of ZFPs defined the particular application as outlined above. The binding properties of the ZFPs must be such to ensure both binding specificity and binding stability.

In this case, the nucleic acid target material is double stranded (ds) DNA. The dsDNA target can be either natural genomic DNA, PCR products or synthetic duplexes. Preferably, the dsDNA may be stripped of all cellular or otherwise contaminating proteins. This can be accomplished using any one of the standard methods in the art. For example, genomic DNA is first treated with the enzyme Proteinase K in a buffer containing 10 mM Tris-Cl (pH 7.8), 10 mM EDTA and 0.5% SDS. After incubation at 37° C. for approximately 30 minutes, the sample is treated with ECTA to chelate the endogenous Ca2+ and extracted with a solution of phenol:CHCI₃ (1:1) two times to remove all residual protein and protein fragments. The DNA sample is then used directly or concentrated by ethanol precipitation. We also envision that simpler, one-step methods of protein removal, such as boiling the DNA sample for 2 minutes, will also be sufficient to remove endogenous prior to analysis by PBEA.

The protein encoded target molecules will be generated by incubating the dsDNA target with the defined ZFP mixture under buffer conditions that promote stable ZFP binding. These include: standard hybridization conditions with a pH between 4.5 to 9.5, more usually in the range of about 5.5 to 8.5, and preferably in the range of 6 to 8. Various buffers known in the art can be used to achieve the desired pH and maintain the pH during the determination. Illustrative buffers include borate, phosphate, carbonate, Tris, HEPES, barbital and the like. The reaction is conducted for a time sufficient to produce the complete or near complete duplex formation.

The concentrations of the encoding ZFPs and target(s) will vary depending upon the exact application. The number of dsDNA molecules can be as low as a single molecule within a sample mixture but generally may varies from about 10² to 10¹⁴, more usually from about 10⁴ to 10¹³ molecules in a sample, and preferably at least 10⁶. The concentration of encoding ZFPs can be equimolar to that of target but usually about 10 to 10² times more concentrated, and preferably about 10³ to 10⁶ times more concentrated. The limiting ZFP concentration will be dictated by that which is necessary to drive stable binding (t_{1/2} must be greater than the target molecule translocation time) under target-limiting conditions at the desired assay temperature.

The ionic current signature for the protein encoded target molecules is then analyzed using a synthetic nanopore having the appropriate pore diameter fixed within an apparatus like that previously described (U.S. Pat. No. 5,795,782 & U.S. Pat. No. 6,015,714). The sample can be concentrated at the pore entrance using the electrophoretic concentration method described above.

### EXAMPLE 4

### Universal Oligonucleotide Ligation Analysis (UOLA)

For UOLA, X-mer mixture will e synthesized using standard methods (Caruthers M. et al., Methods in Enzymology, 154; 287-313 (1987)). The length of each X-mer within the X-mer mixture may vary from 6 to 18 nucleotides and the sequence composition of the mixture will also vary depending the particular design of the universal reagent described above. The level of 5′ phosphorylation can also be varied to control for ligation efficiency (see discussion below). In addition to the universal bases to encode by length, the X-mers within the X-mer mixture may contain duplex stabilizing modifications such as 2-aminoadenine, 2-thiothymine, C-5 propynyldc, and C-5 propynyl-dU, a minor groove binding moiety (MGB) (Nucleic Acids Research 25: 3718-3723, 1997) or Locked Nucleic Acids (LNAs) modifications (Wengel J. et al., (1999) Nucleosides and Nucleotides, 18 1365-1370, Kvaerno L. and Wengelj., (1999) Chem. Commun., 657-658).

The nucleic acid target material is either single stranded RNA or single-stranded DNA. The single stranded target is generated using one of a number of methods known in the art such as; assymetric PCR, standard PCR followed by digestion of one strand with exonuclease, or in vitro transcription of RNA targets using a phage RNA polymerase. It is preferred that the single stranded target have little or no intramolecular structures (secondary structure). This can be accomplished using the UNA technology described in Example 1.

The conditions for carrying out the ligation reactions are similar to those described in U.S. Pat. No. 6,218,118. In brief, the pH for the medium us usually in the range of about 4.5 to 9.5, more usually in the range of about 5.5 to 8.5, and preferably in the range of about 6 to 8. The reaction is tconducted for a time sufficient to produce the desired ligated product. Generally, the time period for conducting the entire method will be from about 10 to 200 minutes. It is usually desirable to minimize the time period.

The reaction temperature can vary from 0° C. to 95° C. depending upon the type of ligase used, the concentration of target and X-mers and the thermodynamic properties of the X-mers in the mixture. The concentration of the ligase is usually determined empirically. Preferably, a concentration is used that is sufficient to ligate most if not all of the precursor X-mers that specifically hybridrize to the target nucleic acid. The limiting factors are generally reaction time and cost of the reagent. The identity of the ligase can be one of many known in the art and will depend upon reaction temperature employed. These include; T4 DNA ligase, Taq DNA Ligase, E. coli DNA Ligase and the like.

The concentration of each X-mer precursor is adjusted according to its thermostability as discussed in U.S. Pat. No. 6,218,118. The absolute ratio of target to X-mer precursor is to be determined empirically. Importantly, the level of phosphorylation of the 5′ terminus of the X-mer mixture can affect the extent of ligation (overall number of ligated products) and the length of ligation products (value of n). The extent and length of ligation can also be controlled by introducing a modification at the 3′ terminus of the X-mer mixture that blocks ligation. In one approach three sets of X-mer mixtures are used together in a single ligation reaction mixture. The X-mers in the first X-mer mixture possess a 5′ phosphorylated terminus and a 3′ blocked terminus (5′p-y3′) where the X-mers whereas the X-mers in the second X-mer mixture have both 5′ and 3′ hydroxyl termini (5′OH-OH3′). The X-mers in the third mixture will have 5′p-OH3′ and will be present at the lowest concentration of the three. This will result in predominantly three-way ligation products having the form o-o/p-o/p-y. Blocking of the 3′ terminus may be accomplished, for example, by employing a group that cannot undergo condensation, such as, for example, an unnatural group such as a 3′-phosphate, a 3′-terminal dideoxy, a polymer or surface, or other means for inhibiting ligation. This approach has great informational advantages because the three sets can be jointly optimized.

After ligation is completed, the ligated products will be separated from the target by heating the sample to 95° C. for 5 minutes and quick cooled. The shorter ligated products could be purified away from the longer target using any one of a number of gel filtration methods known in the art.

The ionic current signature for the ligated products will then be analyzed using a synthetic nanopore having the appropriate pore diameter fixed within an apparatus like that previously described (U.S. Pat. No. 5,795,782 & U.S. Pat. No. 6,015,714). The sample may be concentrated at the pore entrance using the electrophoretic method described above.

What is claimed is:

1. A method for characterizing a nucleic acid molecule, comprising:
providing a nucleic acid molecule for characterization;
modifying a property of at least one defined local area of the nucleic acid molecule, wherein the defined local area comprises at least two consecutive nucleotides;
contacting the modified nucleic acid with a substrate, the substrate including a detector capable of identifying a characteristic of the nucleic acid molecule and the detector being responsive to the modification of the nucleic acid molecule;
causing the modified nucleic acid molecule to traverse a defined volume on the substrate so that individual nucleotides of the modified nucleic acid molecule interact with the detector in sequential order, whereby data correlating with the modification of the nucleic acid molecule are obtained.

2. The method of claim 1, wherein two more or local areas of the nucleic acid molecule are modified.

3. The method of claim 1, wherein the defined local area comprises no more than fifty consecutive nucleotides.

4. The method of claim 1, wherein the defined local area comprises no more than thirty consecutive nucleotides.

5. The method of claim 1, wherein the defined local area comprises no more than twenty consecutive nucleotides.

6. The method of claim 1, wherein the defined local area comprises no more than ten consecutive nucleotides.

7. The method of claim 1, wherein the property of the local area to be modified is selected from the group consisting of local bulk, local chemistry, local charge, local charge density, and the local electronic current carrying capacity of the molecule.

8. The method of claim 7, wherein the local defined area is modified by increasing the total bulk of the local defined area.

9. The method of claim 7, wherein the local defined area is modified by altering the charge of the defined local area of the nucleic acid molecule.

10. The method of claim 7, wherein the local defined area is modified by altering the charge density of the defined local area of the nucleic acid molecule.

11. The method of claim 7, wherein the local defined area is modified by altering the local electronic current carrying capacity of the defined local area of the nucleic acid molecule

12. The method of claim 1 or 2, wherein the modifying step is accomplished by chemically modifying a nucleotide of defined local area of the nucleic acid molecule.

13. The method of claim 12, wherein the base moiety of the nucleotide is modified.

14. The method of claim 12, wherein the sugar moiety of the nucleotide is modified.

15. The method of claim 12, wherein the phosphate moiety of the nucleotide is modified.

16. The method of claim 12, wherein the chemical modifier comprises a covalently linked tag, said tag capable of generating an identifiable signal upon interaction with the detector.

17. The method of claim 1 or 2, wherein the modifying step is accomplished by non-covalently binding a probe to the nucleic acid molecule.

18. The method of claim 17, wherein the probe comprises an oligonucleotide.

19. The method of claim 18, wherein the oligonucleotide comprises at least one modified nucleotide.

20. The method of claim 17, wherein the probe comprises a plurality of oligonucleotides.

21. The method of claim 20, wherein the oligonucleotides comprise at least one modified nucleotide.

22. The method of claim 17, wherein the probe comprises a sequence specific protein.

23. The method of claim 22, wherein the sequence-specific protein is a Zn²⁺ finger protein.

24. The method of claim 17, wherein the probe comprises a plurality of sequence specific proteins having a plurality of binding specificities.

25. The method of claim 1, wherein the nucleic acid molecule is single stranded.

26. The method of claim 1, wherein the nucleic acid molecule is double stranded.

27. The method of claim 1, wherein the nucleic acid molecule comprises genomic DNA.

28. The method of claim 17, wherein the probe is chosen to bind a specific sequence of a nucleic acid molecule.

29. The method of claim 28 wherein the sequence contains a single nucleotide polymorphism (SNP).

30. A method for characterizing a nucleic acid molecule, said method comprising:
(a) providing;
(i) a sample comprising at least one nucleic acid molecule for characterization; and
(ii) a probe capable of binding to a specific nucleic acid sequence;
(b) combining the sample and the probe under conditions such that the probe binds to nucleic acid molecule to form a nucleic acid:probe complex at locations where the specific nucleic acid sequence is present;
(c) detecting the presence and location of binding in the nucleic acid:probe complex by:
(i) contacting the nucleic acid:probe complex with a substrate, the substrate including a detector capable of identifying a characteristic of a nucleic acid molecule; and
(ii) causing the nucleic acid:probe complex to traverse a defined volume of the substrate so that nucleotides of the nucleic acid interact with the detector in sequential order, whereby data correlating with the presence and/or location of binding are obtained.

31. The method of claim 30 wherein the probe comprises a sequence-specific binding protein.

32. The method of claim 31, wherein the probe comprises at least one Zn²⁺ finger protein.

33. The method of claim 31, wherein the probe comprises at least two Zn finger proteins, and wherein the each of the Zn²⁺ finger proteins comprises a moiety that promotes cooperative binding to DNA.

34. The method of claim 31, wherein the probe comprises a plurality of Zn²⁺ finger proteins.

35. The method of claim 31, wherein the probe comprises dimeric Zn²⁺ finger proteins.

36. The method of claim 30, wherein the probe comprises at least one oligonucleotide.

37. The method of claim 36, wherein the oligonucleotide is a genome-specific oligonucleotide.

38. The method of claim 36, wherein the oligonucleotide is an allele-specific oligonucleotide.

39. The method of claim 36, wherein the probe comprises a set of universal oligonucleotides.

40. The method of claim 36, wherein the oligonucleotide is an mRNA specific oligonucleotide.

41. The method of claim 40, wherein the oligonucleotide comprises a nucleotide sequence complementary to an mRNA splice junction.

42. The method of claim 30, wherein the probe binds to the nucleic acid molecule at multiple sites to form multiple nucleic acid:probe complexes.

43. The method of claim 42, wherein the probe comprises a sequence-specific binding protein.

44. The method of claim 43, wherein the probe comprises at least one Zn²⁺ finger protein.

45. The method of claim 43, wherein the probe comprises at least two Zn²⁺ finger proteins, and wherein the each of the Zn²⁺ finger proteins comprises a moiety that promotes cooperative binding to DNA.

46. The method of claim 43, wherein the probe comprises dimeric Zn²⁺ finger proteins.

47. The method of claim 42, wherein the probe comprises at least one oligonucleotide.

48. The method of claim 47, wherein the oligonucleotide is a genome-specific oligonucleotide.

49. The method of claim 47, wherein the oligonucleotide is an allele-specific oligonucleotide.

50. The method of claim 47, wherein the probe comprises a set of universal oligonucleotides.

51. The method of claim 47, wherein the oligonucleotide is a mRNA specific oligonucleotide.

52. The method of claim 51, wherein the oligonucleotide comprises a nucleotide sequence corresponding to an mRNA splice junction.

53. A method of characterizing a nucleic acid molecule, the method comprising:
(a) providing a sample containing at least one nucleic acid molecule for characterization;
(b) modifying a property of a plurality of defined local areas of the nucleic acid molecule under conditions wherein the connectivity of the nucleic acid molecule is maintained, wherein the defined local area comprises one or more nucleotides; and
(c) detecting the presence and locations of the defined local areas under conditions wherein the connectivity of the nucleic acid molecule is maintained, whereby data correlating with the presence and/or locations of defined local areas are obtained.

54. The method of claim 53, wherein the modifying step is accomplished by increasing the cross sectional volume of the defined local areas of the nucleic acid molecule.

55. The method of claim 53, wherein the modifying step is accomplished by chemically modifying at least one nucleotide in each of the defined local areas of the nucleic acid molecule.

56. The method of claim 55, wherein the chemical modifier comprises a covalently linked tag, said tag capable of generating an identifiable signal upon interaction with the detector.

57. The method of claim 53, wherein the modifying step is accomplished by non-covalently binding a probe to the nucleic acid molecule.

58. The method of claim 53, wherein the probe comprises an oligonucleotide.

59. The method of claim 58, wherein the oligonucletide comprises at least one modified nucleotide.

60. The method of claim 53, wherein the probe comprises a sequence-specific protein.

61. The method of 60, wherein the sequence-specific protein is a Zn2+ finger protein.

62. The method of claim 53, further providing a plurality of probes, each probe capable of binding to or modifying a specific nucleic acid sequence, and combining the sample and the plurality of probes under conditions wherein the connectivity of the nucleic acid molecule is maintained and wherein the probes bind to the nucleic acid to form nucleic acid: probe complexes at multiple sites on the nucleic acid molecule where the specific nucleic acid sequence is present; and
detecting the presence and locations of probe:nucleic acid complexes under conditions wherein the connectivity of the nucleic acid molecule is maintained.

63. The method of claim 62, wherein the probes comprise Zn²⁺ finger proteins.

64. The method of claim 62, wherein the probes comprise at least two Zn²⁺ finger proteins, and wherein the each of the Zn²⁺ finger proteins comprises a sequence that promotes binding to DNA.

65. The method of claim 62, wherein the probes comprise a plurality of Zn²⁺ finger proteins.

66. The method of claim 62, wherein the probes comprise dimeric Zn²⁺ finger proteins.

67. The method of claim 63, wherein the probes comprise oligonucleotides.

68. The method of claim 66, wherein the oligonucleotides are genome-specific oligonucleotides.

69. The method of claim 66, wherein the oligonucleotides are allele-specific oligonucleotides.

70. The method of claim 66, wherein the oligonucleotides are a set of universal oligonucleotides.

71. A method for characterizing a nucleic acid molecule, comprising:
generating a population of nucleic acid fragments from a target double stranded nucleic acid;
characterizing the nucleic acid fragments by:
(a) contacting the nucleic acid fragment population with a surface, the surface including a detector capable of detecting the interaction time of nucleic acids;
(b) causing the nucleic acid fragments to traverse a defined volume on the surface so that the nucleotides of a nucleic acid fragment interact with the detector in sequential order, whereby data correlated with a characteristic of the nucleic acid fragment are obtained.

72. The method of claim 71, wherein the step of characterizing the nucleic acid fragments includes determining the relative amount and/or length of the fragments.

73. The method of claim 71, wherein the population of nucleic acid fragments are characterized to provide a size distribution of nucleic acid fragments.

74. The method of claim 71, further comprising:
providing at least one probe capable of binding to a specific nucleic acid sequence;
combining the nucleic acid fragments and the probe under conditions such that the probe binds to the nucleic acid fragments to form a nucleic acid: probe complex at locations where the specific nucleic acid sequence is present;
wherein the detector is capable of identifying a characteristic of a nucleic acid fragment;
and wherein data correlating with the presence and/or location of binding to the nucleic acid fragments are obtained.

75. The method of claim 74, wherein the probe comprises a sequence-specific binding protein.

76. The method of claim 75, wherein the probe comprises at least one Zn²⁺ finger protein.

77. The method of claim 75, wherein the probe comprises a plurality of Zn²⁺ finger proteins.

78. The method of claim 75, wherein the probe comprises at least two Zn²⁺ finger proteins, and wherein the each of the Zn²⁺ finger proteins comprises a moiety that promotes cooperative binding to DNA.

79. The method of claim 75, wherein the probe comprises at least one dimer of two Zn²⁺ finger proteins.

80. The method of claim 75, wherein the probe comprises at least one oligonucleotide.

81. The method of claim 80, wherein the oligonucleotide is a genome-specific oligonucleotide.

82. The method of claim 80, wherein the oligonucleotide is an allele-specific oligonucleotide.

83. The method of claim 80, wherein the probe comprises a set of universal oligonucleotides.

84. The method of claim 80, wherein the oligonucleotide is an mRNA specific oligonucleotide.

85. The method of claim 84, wherein the oligonucleotide comprises a nucleotide sequence complementary to an mRNA splice junction.

86. The method of claim 1, 30 or 53, whereby the detector is an electrode, and electron current is detected as the monomers traverse the electrode.

87. The method of claim 86, wherein the defined volume of the substrate is a groove on the substrate surface, and the detector is at a position along the groove such that it locally contacts the nucleic acid as it traverses the groove.

88. The method of claim 1, 30 or 53, wherein the defined volume comprises an aperture located in the substrate, wherein the aperture includes an entry port and an exit port defining a channel there between.

89. The method of claim 88, wherein nucleic acid molecule interactions with the aperture are detected as electronic currents at first and second electrodes adjacent to the aperture and in electrical communication with said channel.

90. The method of claim 89, wherein current along the length of the channel is detected.

91. The method of claim 1, 30 or 53, wherein nucleic acid molecule interactions with the aperture are detected by measuring ionic conductance in the channel.

92. The method of claim 1, 30 or 53, wherein the nucleic acid molecule is caused to traverse the aperture by application of a voltage gradient.

93. The method of claim 1, 30 or 53, wherein the amplitude or duration of individual conductance measurements is indicative of sequential identity of monomers of the polymer molecule.

94. The method of claim 1, 30 or 53, wherein the number of changes in the conductance measurement is an indication of the number of modifications of the cross-sectional volume in the nucleic acid molecule.

95. The method of claim 1, 30 or 53, wherein the duration of the individual conductance measurement is an indication of the number of nucleotides in the polymer molecule.

96. A method for characterizing a nucleic acid molecule, comprising:
providing a nucleic acid molecule for characterization;
modifying an electronic property of the nucleic acid molecule, wherein at least one nucleotide is modified;
contacting the modified nucleic acid with a substrate, the substrate including a detector capable of identifying an electronic property of the nucleic acid molecule and the detector being responsive to modification of the electronic property of the nucleic acid molecule;
causing the modified nucleic acid molecule to traverse a defined volume on or within the substrate, so that individual nucleotides of the modified nucleic acid molecule interact with the detector in sequential order, whereby data correlating with the modification of the electronic property of the nucleic acid molecule are obtained.

97. The method of claim 96, wherein the detector identifies a current tunneling property of the nucleic acid molecule.

98. A method for characterizing a nucleic acid molecule, the method comprising determining a property of the nucleic acid molecule selected from the group consisting of: determining the presence or absence of a single nucleic acid sequence in the nucleic acid molecule, determining the presence or absence of a set of nucleic acid sequences in the nucleic acid molecule, determining the distances between individual sequences in a set of nucleic acid sequences in the nucleic acid molecule, and determining the frequency at which a nucleic acid sequence is present in a nucleic acid molecule, wherein the nucleic acid sequence or set of nucleic acid sequences is present on a single contiguous nucleic acid molecule.
